(19) United States
(12) Patent Application Publication
(10) Pub. No.: US 2009/0137623 A1
(43) Pub. Date: May 28, 2009

(54) MUSCARINIC RECEPTOR ANTAGONISTS

(76) Inventors: Naresh Kumar, Gurgaon (IN); Ian Anthony Cliffe, Gurgaon (IN); Mohammad Salman, Princeton, NJ (US); Venkata P. Palle, Pune (IN); Kirandeep Kaur, Gurgaon (IN); Yogesh D. Shejul, Ahmednagar (IN); Anita Chugh, New Delhi (IN); Suman Gupta, Gurgaon (IN); Abhijit Ray, New Delhi (IN); Shivani Malhotra, New Delhi (IN); Raj Kumar Shirumalla, New Delhi (IN)

Correspondence Address: RANBAXY INC., 600 COLLEGE ROAD EAST, SUITE 2100, PRINCETON, NJ, 08540, US

(21) Appl. No.: 12/158,435

(22) PCT Filed: Jan. 2, 2007
(86) PCT No.: PCT/IB07/50003

§ 371 Date: Sep. 25, 2008

(30) Foreign Application Priority Data
Dec. 30, 2005 (IN) 3522/DEL/2005

(51) Int. Cl. A61K 31/439; C07D 487/08; A61P 1/00; A61P 3/10; A61P 3/04; A61P 11/06

(52) U.S. Cl. 514/299; 546/112

(57) ABSTRACT

The present invention relates to muscarinic receptor modulators, specifically, 7-oxo-2-azabicyclo[2.2.1]heptanes of formula (I) which are useful for the treatment of various diseases and conditions, for example, Alzheimer's disease, glaucoma, psychosis, particularly schizophrenia or schizophreniform conditions, mania, pain, bipolar disorder, depression, sleeping disorders, epilepsy, gastrointestinal motility disorders, urinary incontinence, and cognition enhancement.

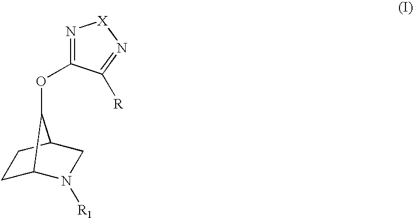

### FIELD OF THE INVENTION

This present invention generally relates to muscarinic receptor antagonists, which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### BACKGROUND OF THE INVENTION

Physiological effects elicited by the neurotransmitter acetylcholine are mediated through its interaction with two major classes of acetylcholine receptors—the nicotinic and muscarinic acetylcholine receptors. Muscarinic receptors belong to the superfamily of G-protein coupled receptors and five molecularly distinct subtypes are known to exist (M₁, M₂, M₃, M₄ and M₅).

These receptors are widely distributed on multiple organs and tissues and are critical to the maintenance of central and peripheral cholinergic neurotransmission. The regional distribution of these receptor sub-types in the brain and other organs has been documented. (for example, the M₁ subtype is located primarily in neuronal tissues such as cerebral cortex and autonomic ganglia, the M₂ subtype is present mainly in the heart and bladder smooth muscle, and the M₃ subtype is located predominantly on smooth muscle and salivary glands (Nature, 323, p. 411 (1986); Science, 237, p. 527 (1987)).

A review in Curr. Opin. Chem. Biol., 3, p. 426 (1999), as well as in Trends in Pharmacol. Sci., 22, p. 409 (2001) by Eglen et. al., describes the biological potentials of modulating muscarinic receptor subtypes by ligands in different disease conditions, such as Alzheimer's disease, pain, urinary disease condition, chronic obstructive pulmonary disease, and the like.

The pharmacological and medical aspects of the muscarinic class of acetylcholine agonists and antagonists are presented in a review in Molecules, 6, p. 142 (2001). Birdsall et. al. in Trends in Pharmacol. Sci., 22, p. 215 (2001) has also summarized the recent developments on the role of different muscarinic receptor subtypes using different muscarinic receptor of knock out mice.

Almost all the smooth muscles express a mixed population of M₂ and M₃ receptors. Although the M₂-receptors are the predominant cholinoreceptors, the smaller population of M₃-receptors appears to be the most functionally important as they mediate the direct contraction of these smooth muscles. Muscarinic receptor antagonists are known to be useful for treating various medical conditions associated with improper smooth muscle function, such as overactive bladder syndrome, irritable bowel syndrome and chronic obstructive pulmonary disease. However the therapeutic utility of antimuscarinics has been limited by poor tolerability as a result of treatment related, frequent systemic adverse events such as dry mouth, constipation, blurred vision, headache, somnolence and tachycardia. Thus, there exists a need for novel muscarinic receptor antagonists that demonstrate target organ selectivity.

WO 04/005252 discloses azabicyclo derivatives described as muscarinic receptor antagonists. WO 04/004629, WO 04/052857, WO 04/067510, WO 04/014853, and WO 04/014363 disclose 3,6-disubstituted azabicyclo[3.1.0]hexane derivatives described as useful muscarinic receptor antagonists. WO2004/056811 discloses flaxavate derivatives as muscarinic receptor antagonists. WO2004/056810 discloses xanthene derivatives as muscarinic receptor antagonists. WO2004/056767 discloses 1-substituted-3-pyrrolidine derivatives as muscarinic receptor antagonists. WO2004/089363, WO2004/089898, WO04069835, WO2004/089900 and WO2004/089364 disclose substituted azabicyclohexane derivatives as muscarinic receptor antagonists. WO2005/026121 and WO2005/026121 disclose process for the preparation of azabicyclohexane derivatives. WO2006/018708 discloses pyrrolidine derivatives as muscarinic receptor antagonists. WO06/054162, WO06/016245, WO06/016345, WO06/05282 and WO2006/35303 disclose azabicyclo derivatives as muscarinic receptor antagonists. WO06/032994 discloses amine derivatives as muscarinic receptor antagonists.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991), describes analogues of oxybutynin, synthesis and antimuscarinic activity of some substituted 7-amino-1-hydroxy-5-heptyn-2-ones and related compounds. Bio-Organic Medicinal Chemistry Letters, 15, p. 2093 (2005) describes synthesis and activity of analogues of Oxybutynin and Tolterodine. Chem. Pharm. Bull. 53(4), 437, 2005 discloses thiazole carboxamide derivatives.

The present invention fills the need of muscarinic receptor antagonists useful in the treatment of disease states associated with improper smooth muscle function and respiratory disorders

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula Ia:

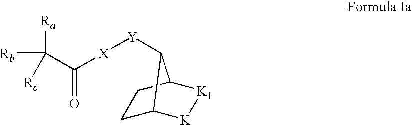

and its pharmaceutically acceptable salts, pharmaceutically acceptable solvates, enantiomers, diastereomers, polymorphs or N-oxides wherein

### K is —CH₂ and K₁ is —NR₁; or K₁ is —CH₂ and K is —NR₁;

Y is alkylene or a single bond;
X is O, S or —NRₛ, (wherein Rₛ, is selected from hydrogen, alkyl, heteroalkyl, aryl, heteroaryl, heterocyclyl, heteroarylalkyl, heterocyclylalkyl, or aralkyl);
Rₐ is hydroxy, alkoxy, alkyl or hydrogen;
R_{b} and R_{c}, are independently selected from alkyl, alkenyl, alkynyl, aryl, cycloalkyl, heteroaryl, heterocyclyl, aralkyl, heterocyclylalkyl or heteroarylalkyl;
R₁ is hydrogen, alkyl, aryl, heteroaryl, cycloalkyl, heterocyclyl, heteroarylalkyl, heterocyclylalkyl, aralkyl, halogen, carboxy, —C(═O)NRₓR_{y}, —COOR₂, —SO₂R₃, or acyl (wherein R₃, Rₓ and R_{y} are defined below);
R₂ is selected from alkyl, aryl, aralkyl, heteroaryl, cycloalkyl, heterocyclyl, heterocyclylalkyl or heteroarylalkyl;
R₃ is alkyl, aralkyl, heteroaryl, heterocyclyl, cycloalkyl, heteroaralkyl, heterocyclylalkyl or NRₓR_{y} (wherein Rₓ and R_{y} are defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also join together to form a heterocyclyl ring.

The following definitions apply to terms as used herein:

The term “alkyl” unless and otherwise specified refers to a monoradical branched or unbranched saturated hydrocarbon chain having from 1 to 20 carbon atoms. Groups such as methyl, ethyl, n-propyl, iso-propyl, n-butyl, iso-butyl, t-butyl, n-hexyl, n-decyl, tetradecyl, and the like exemplify this term. Alkyl may further be substituted with one or more substituents selected from the group consisting of alkenyl, alkynyl, hydroxy, alkoxy, aryloxy, cycloalkyl, acyl, acylamino, acyloxy, —NRₓC(═O)OR₂, azido, cyano, halogen, thioacyl, carboxy, —COOR₂ (wherein R₂ is same as defined above), thiol, alkoxyamino, —NRₓR_{y}, —C(═O)NRₓR_{y}, —OC(═O)NRₓR_{y}, —NRₓC(═O)NRₓR_{y}, (wherein Rₓ and R_{y} are the same as defined earlier), nitro, —S(O)ₙR₃ (wherein R₃ is as defined above and n is 0, 1 or 2). Unless otherwise constrained by the definition, all substituents may be further substituted by 1-3 substituents chosen from alkyl, carboxy, —COOR₂ (wherein R₂ is the same as defined earlier), —NRₓR_{y}, —C(═O)NRₓR_{y}, —OC(═O)NRₓR_{y}, —NRₓC(═O)NRₓR_{y}, —NRₓC(═O)OR₂, (wherein Rₓ and R_{y} are the same as defined earlier), hydroxy, alkoxy, halogen, —CF₃, cyano and —S(O)ₙR₃ (where n and R₃ are the same as defined earlier).

Alkyl group as defined above may also be interrupted by 1-5 atoms of groups independently chosen from oxygen, sulfur and —NRₛ, (where Rₛ, is same as defined earlier)

The term “alkenyl” unless and otherwise specified refers to a monoradical of a branched or unbranched unsaturated hydrocarbon group preferably having 2 to 20 carbon atoms with cis or trans geometry. Preferred alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom.

It may further be substituted with one or more substituents selected from the group consisting of alkyl, alkynyl, alkoxy, acyl, acylamino, acyloxy, —CF₃, —NRₓR_{y}, —C(═O)NRₓR_{y}, —OC(═O)NRₓR_{y}, —NRₓC(═O)NRₓR_{y} (wherein Rₓ and R_{y} are the same as defined earlier), —NRₓC(═O)OR₂, azido, cyano, halogen, hydroxy, acyl, carboxy, —COOR₂ (wherein R₂ is the same as defined earlier), thiol, aryl, aralkyl, aryloxy, cycloalkyl, heterocyclyl, heteroaryl, heterocyclylalkyl, heteroarylalkyl, alkoxyamino, nitro, —S(O)ₙR₃ (wherein n and R₃ are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, cyano, —NRₓR_{y}, —C(═O)NRₓR_{y}, —OC(═O)NRₓR_{y} (wherein Rₓ and R_{y} are the same as defined earlier) and —S(O)ₙR₃ (where R₃ and n are the same as defined earlier).

The term “alkynyl” unless and otherwise specified refers to a monoradical of an unsaturated hydrocarbon, preferably having from 2 to 20 carbon atoms. Preferred alkynyl groups include ethynyl, propargyl or propynyl, and the like. In the event that alkynyl is attached to the heteroatom, the triple bond cannot be alpha to the heteroatom. It may further be substituted with one or more substituents selected from the group consisting of alkyl, alkenyl, alkoxy, cycloalkyl, acyl, acylamino, alkoxyamino, acyloxy, —NRₓC(═O)OR₂, azido, cyano, halogen, hydroxy, thioacyl, —CF₃, carboxy, —COOR₂ (wherein R₂ is the same as defined earlier), thiol, aryl, aralkyl, aryloxy, nitro, heterocyclyl, heteroaryl, heterocyclylalkyl, heteroarylalkyl, —NRₓR_{y}, —C(═O)NRₓR_{y}, —OC(═O)NRₓR_{y}, —NRₓC(═O)NRₓR_{y} (wherein Rₓ and R_{y} are the same as defined earlier), —S(O)ₙR₃ (wherein n and R₃ are the same as defined earlier). Unless otherwise constrained by the definition, all substituents may optionally be further substituted by 1-3 substituents chosen from alkyl, carboxy, —COOR₂ (wherein R₂ is the same as defined earlier), hydroxy, alkoxy, halogen, —CF₃, —NRₓR_{y}, —C(═O)NRₓR_{y}, —OC(═O)NRₓR_{y} (wherein Rₓ and R_{y} are the same as defined earlier), cyano and —S(O)ₙR₃ (wherein R₃ and n are the same as defined earlier).

The term “alkoxy” denotes the group O-alkyl wherein alkyl is the same as defined above.

The term “aryl” herein refers to a carbocyclic aromatic group, for example, phenyl, biphenyl or naphthyl ring and the like optionally substituted with 1 to 3 substituents selected from the group consisting of halogen (F, Cl, Br, I), hydroxy, alkyl, alkenyl, alkynyl, aryl, aralkyl, cycloalkyl, heterocyclyl, heteroaryl, heterocyclylalkyl or heteroarylalkyl, alkoxy, aryloxy, —CF₃, nitro, —NRₓR_{y}, acyl, cyano, acylamino, thioacyl, —C(═O)NRₓR_{y}, —C(═NOH)NH₂, —NRₓC(═O)NRₓR_{y}, —OC(═O)NRₓR_{y} (wherein Rₓ and R_{y} are the same as defined earlier), carboxy, —S(O)ₙR₃ (where R₃ and n are the same as defined earlier), —COOR₂ (wherein R₂ is the same as defined earlier), —NRₓC(═O)OR₂. The aryl group may also be fused with a heterocyclic ring, heteroaryl or cycloalkyl ring.

The term “aralkyl” refers to aryl linked through alkyl (wherein alkyl is the same as defined above) portion and the said alkyl portion contains carbon atoms from 1-6 and aryl is as defined above.

The term “cycloalkyl” refers to cyclic alkyl groups containing 3 to 20 carbon atoms having a single cyclic ring or multiple condensed rings, which may optionally contain one or more olefinic bonds, unless or otherwise constrained by the definition. Such cycloalkyl groups include, by way of example, single ring structures such as cyclopropyl, cyclobutyl, cyclopentyl, cyclohexyl, cycloheptyl, cyclooctyl, cyclopentenyl, and the like, or multiple ring structures such as adamantanyl, and bicyclo[2.2.1]heptane, or cyclic alkyl groups to which is fused with an aryl group, for example indane or tetrahydro-naphthalene and the like.

It may further be substituted with one or more substituents selected from the group consisting of alkyl, alkenyl, alkynyl, alkoxy, cycloalkyl, acyl, acylamino, acyloxy, aryl aralkyl,

—NRₓC(═O)OR₂, azido, cyano, halogen, hydroxy, thioacyl, carboxy, —COOR₂ (wherein R₂ is the same as defined earlier), thiol, aryl, aralkyl, aryloxy, —NRₓR_{y}, —NRₓC(═O)NRₓR_{y}, —C(═O)NRₓR_{y}, —OC(═O)NRₓR_{y} (wherein Rₓ and R_{y} are the same as defined earlier), nitro, heterocyclyl, heteroaryl, heterocyclylalkyl, heteroarylalkyl, —CF₃, —S(O)ₙR₃ (wherein R₃ and n are the same as defined earlier). Unless otherwise constrained by the definition, all substituents may optionally be further substituted by 1-3 substituents chosen from alkyl, carboxy, hydroxy, alkoxy, halogen, CF₃, —NRₓR_{y}, —C(═O)NRₓR_{y}, NRₓC(═O)NRₓR_{y}, —OC(═O)NRₓR_{y} (wherein Rₓ and R_{y} are the same as defined earlier), cyano and —S(O)ₙR₃ (where R₃ and n are the same as defined earlier).

The term “carboxy” as defined herein refers to —C(═O)OH.

The term “aryloxy” denotes the group O-aryl, wherein aryl is as defined above.

The term “heteroaryl” unless and otherwise specified refers to monocyclic aromatic ring structure containing 5 or 6 carbon atoms, a bicyclic or a tricyclic aromatic group having 8 to 10 carbon atoms, with one or more heteroatom(s) independently selected from the group consisting of N, O and S optionally substituted with 1 to 3 substituent(s) selected from the group consisting of halogen (F, Cl, Br, I), hydroxy, alkoxy, alkyl, alkenyl, alkynyl, cycloalkyl, heterocyclyl, aryl, aralkyl, heteroaryl, heterocyclylalkyl, heteroarylalkyl, acyl, acylamino, thioacyl, alkoxyamino, —NRₓC(═O)OR₂, carboxy, —S(O)ₙR₃ (where R₃ and n are the same as defined earlier), —CF₃, —COOR₂ (wherein R₂ is the same as defined earlier), cyano, nitro, —NRₓR_{y}, —C(═O)NRₓR_{y}, —NRₓC(═O)NRₓR_{y} and —OC(═O)NRₓR_{y} (wherein Rₓ and R_{y} are the same as defined earlier). Examples of heteroaryl groups are pyridinyl, pyridazinyl, pyrimidinyl, pyrrolyl, oxazolyl, thiazolyl, thienyl, isoxazolyl, triazinyl, furanyl, pyrazolyl, imidazolyl, benzimidazolone, pyrazolone, benzofuranyl, indolyl, benzothiazolyl, xanthene, benzoxazolyl, and the like.

The term “heterocyclyl” unless and otherwise specified refers to a non aromatic monocyclic, bicyclic (fused, bridged, or spiro) or tricyclic cycloalkyl group having 5 to 10 atoms in which 1 to 3 carbon atoms in a ring are replaced by heteroatoms selected from the group comprising of O, S and N, and are optionally benzofused or fused heteroaryl of 5-6 ring members and the said heterocyclyl group is optionally substituted wherein the substituents are selected from the group consisting of halogen (F, Cl, Br, I), hydroxy, alkoxy, alkyl, alkenyl, alkynyl, cycloalkyl, heterocyclyl, aryl, heteroaryl, aralkyl, heterocyclylalkyl, heteroarylalkyl, acyl, acylamino, thioacyl, cyano, alkoxyamino, —NRₓC(═O)OR₂, nitro, —CF₃, carboxy, —S(O)ₙR₃ (where R₃ and n are the same as defined earlier), —COOR₂ (wherein R₂ is the same as defined earlier), —NRₓC(═O)NRₓR_{y}, —C(═O)NRₓR_{y}, —OC(═O)NRₓR_{y} (wherein Rₓ and R_{y} are the same as defined earlier). Examples of heterocyclyl groups are tetrahydrofuranyl, dihydrofuranyl, dihydropyridinyl, isoxazolinyl, piperidinyl, morpholine, piperazinyl, dihydrobenzofuryl, azabicyclohexyl, azabicyclooctyl, dihydroindolyl, and the like.

“Heteroarylalkyl” refers to heteroaryl (wherein heteroaryl is same as defined earlier) linked through alkyl (wherein alkyl is the same as defined above) portion and the said alkyl portion contains carbon atoms from 1-6.

“Heterocyclylalkyl” refers to heterocyclyl (wherein heterocyclyl is same as defined earlier) linked through alkyl (wherein alkyl is the same as defined above) portion and the said alkyl portion contains carbon atoms from 1-6.

“Acyl” refers to —C(═O)R″ wherein R″ is selected from the group hydrogen, alkyl, alkenyl, alkynyl, cycloalkyl, aryl, aralkyl, heteroaryl, heterocyclyl, heteroarylalkyl or heterocyclylalkyl.

“Thioacyl” refers to —C(═S)H or —C(═S)R″ wherein R″ is selected is the same as defined earlier.

The term “leaving group” generally refers to groups that exhibit the desirable properties of being labile under the defined synthetic conditions and also, of being easily separated from synthetic products under defined conditions. Examples of such leaving groups includes but not limited to halogen (F, Cl, Br, I), triflates, tosylate, mesylates, alkoxy, thioalkoxy, hydroxy radicals and the like.

The term “Protecting Groups” is used herein to refer to known moieties, which have the desirable property of preventing specific chemical reaction at a site on the molecule undergoing chemical modification intended to be left unaffected by the particular chemical modification. Also the term protecting group, unless or other specified may be used with groups such as hydroxy, amino, carboxy and example of such groups are found in T. W. Greene and P. G. M. Wuts, “Protective Groups in Organic Synthesis”, 2^{nd} Edn. John Wiley and Sons, New York, N.Y., which is incorporated herein by reference. The species of the carboxylic protecting groups, amino protecting groups or hydroxy protecting group employed is not so critical so long as the derivatised moiety/moieties is/are stable to conditions of subsequent reactions and can be removed at the appropriate point without disrupting the remainder of the molecule.

The term “pharmaceutically acceptable salts” refers to derivatives of compounds that can be modified by forming their corresponding acid or base salts. Examples of pharmaceutically acceptable salts include, but are not limited to, mineral or organic acids salts of basic residues (such as amines), or alkali or organic salts of acidic residues (such as carboxylic acids), and the like. Pharmaceutically acceptable salts may also be formed by complete derivatization of the amine moiety e.g. quaternary ammonium salts.

In accordance with a second aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder associated with muscarinic receptors, comprising administering to a patient in need thereof, an effective amount of a muscarinic receptor antagonist compound as described above.

In accordance with a fourth aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fifth aspect, there are provided processes for preparing the compounds as described above.

The compounds described herein exhibit significant potency in terms of their activity, as determined by in vitro receptor binding and functional assays and in vivo experiments using anaesthetized rabbits. The compounds that were found active in vitro were tested in vivo. Some of the compounds are potent muscarinic receptor antagonists with high affinity towards M₃ than M₂ and/or M₅ receptors. Therefore, pharmaceutical compositions for the possible treatment for the disease or disorders associated with muscarinic receptors are provided. In addition, the compounds can be administered orally or parenterally.

### DETAILED DESCRIPTION OF THE INVENTION

The compounds disclosed herein may be prepared by methods represented by the reaction sequences, for example, as generally shown in Scheme I

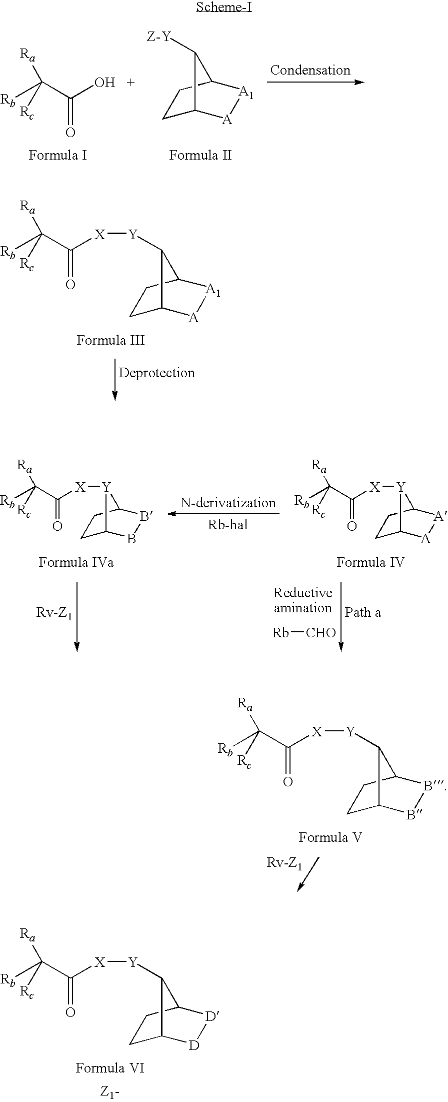

The compounds of Formulae III and IV can be prepared, for example, by following the reaction sequences as shown in the preparative scheme I. The preparation comprises condensing a compound of Formula I (wherein Rₐ, R_{b} and R_{c} are the same as defined above) with a compound of Formula II (wherein A is —CH₂ and A₁ is —NP; or A₁ is —CH₂ and A is —NP; Y is the same as defined earlier; Z is a leaving group (for example, hal (Cl, Br or I), —Omesyl, —Otosyl or —Otriflyl), —SH or —NH₂ and P is a protecting group, for example, aralkyl, —C(═O)Oaralkyl, —C(═O)OC(CH₃)₃, —C(═O)OC(CH₃)₂CHBr₂ or —C(═O)OC(CH₃)₂CCl₃) to give a compound of Formula III (wherein A is —CH₂ and A₁ is —NP; or A₁ is —CH₂ and A is —NP; Y is the same as defined earlier; X is the same as defined earlier), which is deprotected to give the compound of Formula IV (wherein A′ is —CH₂ and A″ is —NH; or A″ is —CH₂ and A′ is —NH; Y is the same as defined earlier; which undergoes N-derivatization to give a compound of Formula IVa (wherein B is —CH₂ and B′ is —NR_{b} or B′ is —CH₂ and B is —NR_{b}; Y is the same as defined earlier). The compound of Formula IV (path a) undergoes reductive amination with a compound of Formula R_{b}—CHO to give a compound of Formula V (wherein B″ is —CH₂ and B′″ is —NCH₂R_{b}; or B′″ is —CH₂ and B″ is —NCH₂R_{b}; Y is the same as defined earlier). The compound of Formula V and IV a can be reacted with a compound of Formula Rv-Z1 (wherein Rv is alkyl, alkenyl, alkynyl, aryl, cycloalkyl, heteroaryl, heterocyclyl, heterocyclylalkyl, heteroarylalkyl or aralkyl and Z1 is an anion disclosed in International Journal of pharmaceutics, 33 (1986), page 202, for example, but not limited to, acetate, tartarate, chloride, bromide, iodide, sulphate, phosphate, nitrate, carbonate, fumarate, glutamate, citrate, methanesulphonate, toluenesulphonate, benzenesulphonate, maleate or succinate) to give a compound of Formula VI (wherein D is —CH₂ and D′ is

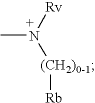

### or D′ is —CH₂ and D is

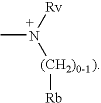

The condensation of the compound of Formula I with Compound of Formula II [when Z is hal (Cl, Br, I)] can be carried out in an organic solvent (for example, toluene, heptane or xylene) in the presence of a base (for example, 1,8-diazabicyclo[5.4.0]undecen-7-ene or 1,4-diazabicyclo[2.2.2]octane) to give the compound of Formula III (wherein X is —O or —SH).

The condensation of compound of Formula I with a compound of Formula II (when Z is —NH₂) can be carried out in an organic solvent (for example, dimethylformamide, dichloromethane, chloroform, tetrahydrofuran, dioxane or diethylether) in presence of a base (for example, N-methylmorpholine, triethylamine, diisopropylethylamine or pyridine) with a condensing agent (for example, 1-(3-dimethylaminopropyl)-3-ethylcarbodiimide hydrochloride (EDC.HCl) or dicyclohexylcarbodiimide (DCC) to give the compound of Formula III (wherein X is —NRₛ).

The deprotection of a compound of Formula III (wherein P can be aralkyl) to give a compound of Formula IV can be carried out in an organic solvent (for example, methanol, ethanol, propanol or isopropylalcohol) in the presence of a deprotecting agent (for example, palladium on carbon in presence of hydrogen gas or palladium on carbon in ammonium formate solution).

The deprotection of a compound of Formula III (wherein P can be —C(═O)Oaralkyl) to give a compound of Formula IV can be carried out with alkaline (for example, potassium hydroxide, sodium hydroxide or lithium hydroxide) solution of an alcohol (for example, methanol, ethanol propanol, diethylether or isopropylalcohol). Alternatively, The deprotection of a compound of Formula III (when P is —C(═O)Oaralkyl) can be carried out in an organic solvent (for example, methanol, ethanol, propanol or isopropylalcohol) in the presence of a deprotecting agent (for example, palladium on carbon in presence of hydrogen gas or palladium on carbon with a source of hydrogen gas (for example, ammonium formate solution, cyclohexene or formic acid)).

The deprotection of a compound of Formula III (wherein P can be —C(═O)OC(CH₃)₃ or —C(═O)OC(CH₃)₂CHBr₂) to give a compound of Formula IV can be carried out in an acidic solution of an alcohol (for example, hydrochloric acid solution of methanol, ethanol, propanol, isopropylalcohol, ethylacetate or ether) or trifluoroacetic acid in dichloromethane.

The deprotection of a compound of Formula III (wherein P can be —C(═O)OC(CH₃)₂CCl₃) to give a compound of Formula IV can be carried out by a supernucleophile (for example, lithium cobalt (I) phthalocyanine, zinc and acetic acid or cobalt phthalocyanine).

The N-derivatization of a compound of Formula IV with a compound R_{b}-hal to give a compound of Formula IVa can be carried out in an organic solvent (for example, acetonitrile, dichloromethane, chloroform or carbon tetrachloride) in the presence of a base (for example, potassium carbonate, sodium carbonate or sodium bicarbonate).

The reductive amination of a compound of Formula IV with Rb—CHO to give a compound of Formula V can be carried out in an organic solvent (for example, acetonitrile or dichloromethane or tetrahydrofuran) in the presence of reducing agent (for example, sodium cyanoborohydride or sodium triacetoxyborohydride).

The reaction of a compound of Formula V and IVa with a compound of Formula Rv-Z₁ of give a compound of Formula VI can be carried out in an organic solvent for example, dichloromethane, dichloroethane, carbon tetrachloride, chloroform or acetonitrile.

Particular compounds are mentioned below:
2-Benzyl-2-azabicyclo[2.2.1]hept-7-yl-2-hydroxy(diphenyl)acetate (Compound No. 1),
2-Benzyl-2-azabicyclo[2.2.1]hept-7-yl-2-cyclopentyl(hydroxy)-2-thienylacetate (Compound No. 2),
2-Azabicyclo[2.2.1]hept-7-yl-2-hydroxy(diphenyl)acetate (Compound No. 3),
2-Azabicyclo[2.2.1]hept-7-yl-2-cyclopentyl(hydroxy)phenylacetate (Compound No. 4),
2-Benzyl-2-azabicyclo[2.2.1]hept-7-yl-2-cyclopentyl(hydroxy)phenylacetate (Compound No. 5),
2-Benzyl-2-azabicyclo[2.2.1]hept-7-yl-2-hydroxy(phenyl)-2-thienylacetate (Compound No. 6),
N-(2-benzyl-2-azabicyclo[2.2.1]hept-7-yl)-2,2-diphenylpropanamide (Compound No. 7),
N-(2-benzyl-2-azabicyclo[2.2.1]hept-7-yl)-2-cyclopentyl-2-hydroxy-2-(2-thienyl)acetamide (Compound No. 8),
N-(2-benzyl-2-azabicyclo[2.2.1]hept-7-yl)-2-hydroxy-2-phenyl-2-(2-thienyl)acetamide (Compound No. 9),
N-(2-benzyl-2-azabicyclo[2.2.1]hept-7-yl)-2-cyclopentyl-2-hydroxy-2-phenylacetamide (Compound No. 10),
2-Benzyl-2-azabicyclo[2.2.1]hept-7-yl 2,2-diphenylpropanoate (Compound No. 11),
2-Azabicyclo[2.2.1]hept-7-yl 2,2-diphenylpropanoate (Compound No. 12),
N-(2-benzyl-2-azabicyclo[2.2.1]hept-7-yl)-2-hydroxy-2,2-diphenylacetamide (Compound No. 13),
N-2-azabicyclo[2.2.1]hept-7-yl-2-hydroxy-2,2-diphenylacetamide (Compound No. 14),
N-2-azabicyclo[2.2.1]hept-7-yl-2,2-diphenylpropanamide (Compound No. 15),
N-2-azabicyclo[2.2.1]hept-7-yl-2-cyclopentyl-2-hydroxy-2-phenylacetamide (Compound No. 16),
2-Benzyl-2-azabicyclo[2.2.1]hept-7-yl hydroxy[bis(3-methylphenyl)]acetate (Compound No. 17),
2-Benzyl-2-azabicyclo[2.2.1]hept-7-yl methoxy(diphenyl)acetate (Compound No. 18),
(1R)-2-Azabicyclo[2.2.1]hept-7-yl (2R)-((1R or 1S)-3,3-difluorocyclopentyl)(hydroxy)phenylacetate (Compound No. 19),
(1R or 1S)-2-Azabicyclo[2.2.1]hept-7-yl cyclohexyl(hydroxy)phenylacetate (Compound No. 20),
2-(Pyridin-3-ylmethyl)-(1R or 1S)-2-azabicyclo[2.2.1]hept-7-yl cyclopentyl(hydroxy)phenylacetate (Compound No. 21),
2-(1,3-Benzodioxol-5-ylmethyl)-(1R or 1S)-2-azabicyclo[2.2.1]hept-7-yl cyclopentyl(hydroxy)phenylacetate (Compound No. 22),
2-(Cyclohexylmethyl)-(1R or 1S)-2-azabicyclo[2.2.1]hept-7-yl cyclopentyl(hydroxy)phenylacetate (Compound No. 23),
2-(4-Methylbenzyl)-(1R or 1S)-2-azabicyclo[2.2.1]hept-7-yl cyclopentyl(hydroxy)phenylacetate (Compound No. 24),
2-(2-Fluorobenzyl)-(1R or 1S)-2-azabicyclo[2.2.1]hept-7-yl cyclopentyl(hydroxy)phenylacetate (Compound No. 25),
2-(4-Fluorobenzyl)-(1R or 1S)-2-azabicyclo[2.2.1]hept-7-yl cyclopentyl(hydroxy)phenylacetate (Compound No. 26),
2-(3-Methoxybenzyl)-(1R or 1S)-2-azabicyclo[2.2.1]hept-7-yl cyclopentyl(hydroxy)phenylacetate (Compound No. 27),
2-Methyl-(1R or 1S)-2-azabicyclo[2.2.1]hept-7-yl cyclopentyl(hydroxy)phenylacetate (Compound No. 28),
2-(3-Methylbenzyl)-(1R or 1S)-2-azabicyclo[2.2.1]hept-7-yl cyclopentyl(hydroxy)phenylacetate (Compound No. 29),
2-[(1′R)-1-phenylethyl]-(1S or 1R)-2-azabicyclo[2.2.1]hept-7-yl (2R)-cyclopentyl(hydroxy)phenylacetate (Compound No. 30),
(1S or 1R)-2-Azabicyclo[2.2.1]hept-7-yl (2R)-cyclopentyl(hydroxy)phenylacetate (Compound No. 31),
2-[(1′S)-1-phenylethyl]-(1R or 1S)-2-azabicyclo[2.2.1]hept-7-yl cyclohexyl(hydroxy)phenylacetate (Compound No. 32),
2-[(1′S)-1-phenylethyl]-(1R or 1S)-2-azabicyclo[2.2.1]hept-7-yl cyclopentyl(hydroxy)phenylacetate (Compound No. 33),
2-[(1′S)-1-phenylethyl]-(1R or 1S)-2-azabicyclo[2.2.1]hept-7-yl (2R)-[(1S or 1R) (3,3-difluorocyclopentyl)(hydroxy)phenylacetate (Compound No. 34),
2-[(1′S)-1-phenylethyl]-(1R or 1S)-2-azabicyclo[2.2.1]hept-7-yl (2R)-[(1R or 1S) (3,3-difluorocyclopentyl)](hydroxy)phenylacetate (Compound No. 35),
2-[(1′S)-1-phenylethyl]-(1R or 1S)-2-azabicyclo[2.2.1]hept-7-yl (2R)-cyclopentyl(hydroxy)phenylacetate (Compound No. 36),
(1R or 1S)-2-Azabicyclo[2.2.1]hept-7-yl (2R)-cyclopentyl(hydroxy)phenylacetate (Compound No. 37),
(1R or 1S)-2-Azabicyclo[2.2.1]hept-7-yl cyclopentyl(hydroxy)phenylacetate (Compound No. 38),
(1R or 1S)-7-{[(2R)-2-cyclopentyl-2-hydroxy-2-phenylacetyl]oxy}-2,2-dimethyl-2-azoniabicyclo[2.2.1]heptane Iodide (Compound No. 39)
7-{[cyclopentyl(hydroxy)phenylacetyl]oxy}-(1R or 1S)-2-(4-fluorobenzyl)-2-methyl-2-azoniabicyclo[2.2.1]heptane Iodide (Compound No. 41)
7-{[cyclopentyl(hydroxy)phenylacetyl]oxy}-(1R or 1S)-2-(2-fluorobenzyl)-2-methyl-2-azoniabicyclo[2.2.1]heptane Iodide (Compound No. 42)
7-{[cyclopentyl(hydroxy)phenylacetyl]oxy}-(1R or 1S)-2-methyl-2-(4-methylbenzyl)-2-azoniabicyclo[2.2.1]heptane Iodide (Compound No. 43)
2-(cyclohexylmethyl)-7-{[cyclopentyl(hydroxy)phenylacetyl]oxy}-(1R or 1S)-2-methyl-2-azoniabicyclo[2.2.1]heptane Iodide (Compound No. 44)
7-{[(2R)-2-cyclopentyl-2-hydroxy-2-phenylacetyl]oxy}-(1S or 1R)-2,2-dimethyl-2-azoniabicyclo[2.2.1]heptane Iodide (Compound No. 45)
7-[2-cycloheptyl(hydroxy)-2-thienylacetoxy]-2-methyl-2-[(1′S)-1-phenylethyl]-(1R or 1S)-2-azoniabicyclo[2.2.1]heptane Iodide (Compound No. 46)
7-{[hydroxy(phenyl)-2-thienylacetyl]oxy}-2-methyl-2-[(1′S)-1-phenylethyl]-(1R or 1S)-2-azoniabicyclo[2.2.1]heptane Iodide (Compound No. 47)
7-[2-cycloheptyl(hydroxy)phenylacetoxy]-2-methyl-2-[(1′S)-1-phenylethyl]-(1R or 1S)-2-azoniabicyclo[2.2.1]heptane Iodide (Compound No. 48)
7-[2-hydroxy(diphenyl)acetoxy]-2-methyl-2-[(1′S)-1-phenylethyl]-(1R or 1S)-2-azoniabicyclo[2.2.1]heptane Iodide (Compound No. 49)
7-[2-hydroxy(4-methylphenyl)phenylacetoxy]-2-methyl-2-[(1′S)-1-phenylethyl]-(1R or 1S)-2-azoniabicyclo[2.2.1]heptane Iodide (Compound No. 50)
7-[2-(4-fluorophenyl)(hydroxy)phenylacetoxy]-2-methyl-2-[(1′S)-1-phenylethyl]-(1R or 1S)-2-azoniabicyclo[2.2.1]heptane Iodide (Compound No. 51)
7-{[cyclopentyl(phenyl)acetyl]oxy}-2-methyl-2-[(1′S)-1-phenylethyl]-(1R or 1S)-2-azoniabicyclo[2.2.1]heptane Iodide (Compound No. 52)
7-({(2R)-2-[(1S)-3,3-difluorocyclohexyl]-2-hydroxy-2-phenylacetyl}oxy)-2-methyl-2-[(1′S)-1-phenylethyl]-(1R or 1S)-2-azoniabicyclo[2.2.1]heptane iodide (Compound No. 53)
7-{[(2R)-2-cyclopentyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 54)
7-{[(2R or 2S)-2-cycloheptyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 55)
2-(1,2,3-benzotrioxol-5-ylmethyl)-7-[(2-cycloheptyl-2-hydroxy-2-phenylacetyl)oxy]-(1R or 1S)-2-methyl-2-azoniabicyclo[2.2.1]heptane Bromide (Compound No. 56)
7-[2-cycloheptyl(hydroxy)phenylacetoxy]-2-ethyl-(1R or 1S)-2-methyl-2-azoniabicyclo[2.2.1]heptane Iodide (Compound No. 57)
7-[2-cycloheptyl(hydroxy)phenylacetoxy]-(1R or 1S)-2-methyl-2-propyl-2-azoniabicyclo[2.2.1]heptane Iodide (Compound No. 58)
7-[2-cycloheptyl(hydroxy)phenylacetoxy]-(1R or 1S)-2-(4-fluorobenzyl)-2-methyl-2-azoniabicyclo[2.2.1]heptane Bromide (Compound No. 59)
7-[2-cycloheptyl(hydroxy)phenylacetoxy]-2-(4-methoxybenzyl)-(1R or 1S)-2-methyl-2-azoniabicyclo[2.2.1]heptane Chloride (Compound No. 60)
7-{[cycloheptyl(hydroxy)phenylacetyl]oxy}-(1R or 1S)-2-methyl-2-(2-methylprop-2-en-1-yl)-2-azoniabicyclo[2.2.1]heptane Bromide (Compound No. 61)
2-benzyl-7-[2-cycloheptyl(hydroxy)phenylacetoxy]-(1R or 1S)-2-methyl-2-azoniabicyclo[2.2.1]heptane Bromide (Compound No. 62)
7-[2-cycloheptyl(hydroxy)phenylacetoxy]-(1R or 1S)-2-methyl-2-(3-phenylpropyl)-2-azoniabicyclo[2.2.1]heptane Bromide (Compound No. 63)
7-({(2R)-2-[(1R)-3,3-difluorocyclopentyl]-2-hydroxy-2-phenylacetyl}oxy)-(1R or 1S)-2-methyl-2-propyl-2-azoniabicyclo[2.2.1]heptane Bromide (Compound No. 64)
7-({(2R)-2-[(1R)-3,3-difluorocyclopentyl]-2-hydroxy-2-phenylacetyl}oxy)-(1R or 1S)-2-methyl-2-(3-phenylpropyl)-2-azoniabicyclo[2.2.1]heptane Bromide (Compound No. 65)
7-({(2R)-2-[(1R)-3,3-difluorocyclopentyl]-2-hydroxy-2-phenylacetyl}oxy)-(1R or 1S)-2-(4-fluorobenzyl)-2-methyl-2-azoniabicyclo[2.2.1]heptane Bromide (Compound No. 66)
7-({(2R)-2-[(1R)-3,3-difluorocyclopentyl]-2-hydroxy-2-phenylacetyl}oxy)-(1R or 1S)-2-methyl-2-(2-methylprop-2-en-1-yl)-2-azoniabicyclo[2.2.1]heptane Bromide (Compound No. 67)
2-(1,3-benzodioxol-5-ylmethyl)-7-({(2R)-2-[(1R)-3,3-difluorocyclopentyl]-2-hydroxy-2-phenylacetyl}oxy)-(1R or 1S)-2-methyl-2-azoniabicyclo[2.2.1]heptane Bromide (Compound No. 68)
7-({(2R)-2-[(1R)-3,3-difluorocyclopentyl]-2-hydroxy-2-phenylacetyl}oxy)-(1R or 1S)-2-(4-methoxybenzyl)-2-methyl-2-azoniabicyclo[2.2.1]heptane Bromide (Compound No. 69)
7-{[(2R)-2-cycloheptyl-2-hydroxy-2-phenylacetyl]oxy}-(1R or 1S)-2,2-dimethyl-2-azoniabicyclo[2.2.1]heptane Iodide (Compound No. 70)
7-[2-cyclohexyl(hydroxy)phenylacetoxy]-2-methyl-2-[(1S)-1-phenylethyl]-(1R or 1S)-2-azoniabicyclo[2.2.1]heptane Iodide (Compound No. 71)
7-({(2R)-2-[(1S)-3,3-difluorocycloheptyl]-2-hydroxy-2-phenylacetyl}oxy)-(1R or 1S)-2,2-dimethyl-2-azoniabicyclo[2.2.1]heptane Iodide (Compound No. 72)
7-[(2,2-diphenylpropanoyl)oxy]-(1R or 1S)-2,2-dimethyl-2-azoniabicyclo[2.2.1]heptane Iodide (Compound No. 73)
7-({(2R)-2-[(1R)-3,3-difluorocyclohexyl]-2-hydroxy-2-phenylacetyl}oxy)-(1R or 1S)-2,2-dimethyl-2-azoniabicyclo[2.2.1]heptane Iodide (Compound No. 74)
7-[2-cycloheptyl(hydroxy)phenylacetoxy]-2-methyl-2-[(1′R)-1-phenylethyl]-(1S or 1R)-2-azoniabicyclo[2.2.1]heptane Iodide (Compound No. 75)
7-({(2R)-2-[(1R)-3,3-difluorocyclopentyl]-2-hydroxy-2-phenylacetyl}oxy)-(1R or 1S)-2,2-dimethyl-2-azoniabicyclo[2.2.1]heptane Iodide (Compound No. 76)
7-[2-cycloheptyl(hydroxy)phenylacetoxy]-(1S or 1R)-2,2-dimethyl-2-azoniabicyclo[2.2.1]heptane Iodide (Compound No. 77)
7-{(2R)-2-[(1S)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 78)
7-{(2R)-2-[(1R)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 79)
7-{(2R)-[(1R)-3,3-difluorocyclohexyl](hydroxy)phenylacetoxy}-2-methyl-2-[(1′S)-1-phenylethyl]-(1R or 1S)-2-azoniabicyclo[2.2.1]heptane iodide (Compound No. 80)
(1R or 1S)-2-Methyl-2-azabicyclo[2.2.1]hept-7-yl-(2R)-cyclopentyl(hydroxy)phenylacetate (Compound No. 81) (1S or 1R)-2-Methyl-2-azabicyclo[2.2.1]hept-7-yl-(2R)-cyclopentyl(hydroxy)phenylacetate (Compound No. 82)
(1R or 1S)-2-Methyl-2-azabicyclo[2.2.1]hept-7-yl cyclopentyl(hydroxy)phenylacetate (Compound No. 83)
2-[(1′S)-1-Phenylethyl]-(1R or 1S)-2-azabicyclo[2.2.1]hept-7-yl cycloheptyl(hydroxy)-2-thienylacetate (Compound No. 84)
2-[(1′S)-1-Phenylethyl]-(1R or 1S)-2-azabicyclo[2.2.1]hept-7-yl hydroxy(phenyl)-2-thienylacetate (Compound No. 85)
2-[(1′S)-1-Phenylethyl]-(1R or 1S))-2-azabicyclo[2.2.1]hept-7-yl cycloheptyl(hydroxy)phenylacetate (Compound No. 86)
2-[(1′S)-1-Phenylethyl]-(1R or 1S)-2-azabicyclo[2.2.1]hept-7-yl hydroxy(diphenyl)acetate (Compound No. 87)
2-[(1′S)-1-Phenylethyl]-(1R or 1S)-2-azabicyclo[2.2.1]hept-7-yl hydroxy(4-methylphenyl)phenylacetate (Compound No. 88)
2-[(1′S)-1-Phenylethyl]-(1R or 1S)-2-azabicyclo[2.2.1]hept-7-yl (4-fluorophenyl)(hydroxy)phenylacetate (Compound No. 89)
2-[(1′S)-1-phenylethyl]-(1R or 1S)-2-azabicyclo[2.2.1]hept-7-yl cyclopentyl(phenyl)acetate (Compound No. 90)
2-[(1′S)-1-Phenylethyl]-(1R or 1S)-2-azabicyclo[2.2.1]hept-7-yl-(2R)-[(1S)-3,3-difluorocyclohexyl](hydroxy)phenylacetate (Compound No. 91)
2-[(1′S)-1-phenylethyl]-(1R or 1S)-2-azabicyclo[2.2.1]hept-7-yl (2R)-cyclopentyl(hydroxy)phenylacetate (Compound No. 92)
2-[(1′S)-1-Phenylethyl]-(1R or 1S)-2-azabicyclo[2.2.1]hept-7-yl (2R or 2S)-cycloheptyl(hydroxy)phenylacetate (Compound No. 93)
(1R or 1S)-2-Methyl-2-azabicyclo[2.2.1]hept-7-yl cycloheptyl(hydroxy)phenylacetate (Compound No. 94)
(1R or 1S)-2-Methyl-2-azabicyclo[2.2.1]hept-7-yl (2R)-[(1R)-3,3-difluorocyclopentyl](hydroxy)phenylacetate (Compound No. 95)
2-[(1′S)-1-Phenylethyl]-(1R or 1S)-2-azabicyclo[2.2.1]hept-7-yl cyclohexyl(hydroxy)phenylacetate (Compound No. 96)
(1R or 1S)-2-Methyl-2-azabicyclo[2.2.1]hept-7-yl (2R)-[(1S)-3,3-difluorocyclohexyl](hydroxy)phenylacetate (Compound No. 97)
(1R or 1S)-2-Methyl-2-azabicyclo[2.2.1]hept-7-yl 2,2-diphenylpropanoate (Compound No. 98)
(1R or 1S)-2-Methyl-2-azabicyclo[2.2.1]hept-7-yl (2R)-[(1R)-3,3-difluorocyclohexyl](hydroxy)phenylacetate (Compound No. 99)
(1R or 1S)-2-Methyl-2-azabicyclo[2.2.1]hept-7-yl (2R)-[(1R)-3,3-difluorocyclopentyl](hydroxy)phenylacetate (Compound No. 100)
(1R or 1S)-2-Methyl-2-azabicyclo[2.2.1]hept-7-yl cycloheptyl(hydroxy)phenylacetate (Compound No. 101)
2-[(1′S)-1-Phenylethyl]-(1R or 1S)-2-azabicyclo[2.2.1]hept-7-yl-(2R)-[(1S)-3,3-difluorocyclopentyl](hydroxy)phenylacetate (Compound No. 102)
2-[(1′S)-1-phenylethyl]-(1R or 1S)-2-azabicyclo[2.2.1]hept-7-yl-(2R)-[(1R)-3,3-difluorocyclopentyl](hydroxy)phenylacetate (Compound No. 103)
2-[(1′S)-1-phenylethyl]-(1R or 1S)-2-azabicyclo[2.2.1]hept-7-yl-(2R)-[(1R)-3,3-difluorocyclohexyl](hydroxy)phenylacetate (Compound No. 104)
2-[(1′R)-1-Phenylethyl]-(1S or 1R)-2-azabicyclo[2.2.1]hept-7-yl cycloheptyl(hydroxy)phenylacetate (Compound No. 105)

In the above schemes, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Suitable salts of the compounds represented by the Formula Ia were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, toluenesulphonate and methanesulphonate). When carboxyl groups are included in the Formula Ia as substituents, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula Ia or metabolites, enantiomers, diastereomers, N-oxides, polymorphs, solvates or pharmaceutically acceptable salts thereof, in combination with pharmaceutically acceptable carrier and optionally included excipient can also be produced.

Where desired, the compounds of Formula Ia and/or their pharmaceutically acceptable salts, pharmaceutically acceptable solvates, stereoisomers, tautomers, racemates, prodrugs, metabolites, polymorphs or N-oxides may be advantageously used in combination with one or more other therapeutic agents. Examples of other therapeutic agents, which may be used in combination with compounds of Formula Ia of this invention and/or their pharmaceutically acceptable salts, pharmaceutically acceptable solvates, stereoisomers, tautomers, racemates, prodrugs, metabolites, polymorphs or N-oxides include, but are not limited to, corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

The compositions can be administered by inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients. The compositions can be administered by the nasal respiratory route for local or systemic effect. Compositions can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered nasally from devices, which deliver the formulation in an appropriate manner.

Alternatively, compositions can be administered orally, rectally, parenterally (intravenously, intramuscularly or subcutaneously), intracisternally, intravaginally, intraperitoneally or topically.

Solid dosage forms for oral administration may be presented in discrete units, for example, capsules, cachets, lozenges, tablets, pills, powders, dragees or granules, each containing a predetermined amount of the active compound. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

Solid compositions of a similar type may also be employed as fillers in soft and hard-filled gelatin capsules using such excipients as lactose or milk sugar as well as high molecular weight polyethylene glycols, and the like.

Solid dosage forms can be prepared with coatings and shells, such as enteric coatings and others well known in this art. They may contain opacifying agents, and can also be of such composition that they release the active compound or compounds in a certain part of the intestinal tract in a delayed manner. Examples of embedding compositions which can be used are polymeric substances and waxes.

The active compounds can also be in micro-encapsulated form, if appropriate, with one or more of the above mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, for example, wetting agents, emulsifying and suspending agents, sweetening, flavoring and perfuming agents, colorants or dyes.

Suspensions, in addition to the active compounds, may contain suspending agents, as for example, ethoxylated isostearyl alcohols, polyoxyethylene sorbitol and sorbitan esters, microcrystalline cellulose, aluminium metahydroxide, bentonite, agar-agar and tragacanth, or mixtures of these substances, and the like.

Dosage forms for topical administration of a compound of this invention include powder, spray, inhalant, ointment, creams, salve, jelly, lotion, paste, gel, aerosol, or oil. The active component is admixed under sterile conditions with a pharmaceutically acceptable carrier and any needed preservatives, buffers or propellants as may be required. Opthalmic formulations, eye ointments, powders and solutions are also contemplated as being within the scope of this invention.

Compositions suitable for parenteral injection may comprise pharmaceutically acceptable sterile aqueous or nonaqueous solutions, dispersions, suspensions or emulsions and sterile powders for reconstitution into sterile injectable solutions or dispersions. These preparations may contain anti-oxidants, buffers, bacteriostats and solutes, which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monosterate and gelatin.

Suppositories for rectal administration of the compound of Formula Ia can be prepared by mixing the drug with a suitable nonirritating excipient such as cocoa butter and polyethylene glycols or a suppository wax, which are solid at ordinary temperatures but liquid at body temperature and which therefore melt in the rectum or vaginal cavity and release the drug.

If desired, and for more effective distribution, the compounds can be incorporated into slow release or targeted delivery systems such as polymer matrices, liposomes, and microspheres. They may be sterilized, for example, by filtration through a bacteria-retaining filter, or by incorporating sterilizing agents in the form of sterile solid compositions, which can be dissolved in sterile water, or some other sterile injectable medium immediately before use.

Actual dosage levels of active ingredient in the compositions of the invention and spacing of individual dosages may be varied so as to obtain an amount of active ingredient that is effective to obtain a desired therapeutic response for a particular composition and method of administration. It will be understood, however, that the specific dose level for any particular patient will depend upon a variety of factors including the compound chosen, the body weight, general health, sex, diet, route of administration, the desired duration of treatment, rates of absorption and excretion, combination with other drugs and the severity of the particular disease being treated and is ultimately at the discretion of the physician.

The pharmaceutical compositions described herein can be produced and administered in dosage units, each unit containing a certain amount of at least one compound described herein and/or at least one physiologically acceptable addition salt thereof. The dosage may be varied over extremely wide limits as the compounds are effective at low dosage levels and relatively free of toxicity. The compounds may be administered in the low micromolar concentration, which is therapeutically effective, and the dosage may be increased as desired up to the maximum dosage tolerated by the patient.

The examples mentioned below demonstrate general synthetic procedures, as well as specific preparations of particular compounds. The examples are provided to illustrate the details of the invention and should not be constrained to limit the scope of the present invention.

### EXAMPLES

Various solvents, such as acetone, methanol, pyridine, ether, tetrahydrofuran, hexanes, and dichloromethane, were dried using various drying reagents according to procedures described in the literature. IR spectra were recorded as nujol mulls or a thin neat film on a Perkin Elmer Paragon instrument, Nuclear Magnetic Resonance (NMR) were recorded on a Varian XL-300 MHz or Bruker 400 MHz instrument using tetramethylsilane as an internal standard.

### General Procedure for Preparation of Intermediates

### Synthesis of (1R)-7-bromo-2-(1′ S-phenylethyl)-2-azabicyclo[2.2.1]heptane

### Step a: Synthesis of (1R,1′S)-2-(1′-phenylethyl)-2-azabicyclo(2.2.1)hept-5-ene

The title compound was prepared following the procedure as described in Syn. Comm., 1996, 577-584 wherein to a solution of the compound (S)-methyl benzyl amine (5 g, 0.041 mol) in water (14 ml) was added acetic acid (2.47 g, 0.041 mol) in water (7.1 ml) at 0° C. followed by the addition of cyclopentadiene (0.082 mol) and formaldehyde (0.061 mol) at the same temperature. The reaction mixture was stirred for 20 hours at 0-5° C. The reaction mixture was diluted with water and extracted with hexane. The aqueous layer was cooled at 0° C. followed by the addition of 10% ethyl acetate in hexane (10 ml) solvent mixture. The mixture was basified with aqueous sodium hydroxide solution until pH 9-10 is attained. The aqueous layer was extracted with 10% ethyl acetate in hexane solvent mixture as eluent. The organic layer was dried over anhydrous sodium sulphate and concentrated under reduced pressure to furnish the title compound. Yield: 5.4 g.

### Step b: Synthesis of (1R,2R,3R,4R,6R,1′S)-3-bromo-1-(1′-phenylethyl)-1-azoniatricyclo(2.2.1.0^{2,6})heptyl Bromide

The title compound was prepared following the procedure as described in Helv. Chimica. acta, 76, 1203-1215 (1993).

To a solution of the compound obtained from step a above (0.054 mole, 10.8 g) in dichloromethane was added a solution of bromine in dichloromethane (10.40 g, 25.17 ml, 0.065 mol) at 0° C. The reaction mixture was stirred at 0° C. for 20 hours. The mixture was concentrated under reduced pressure. The residue thus obtained was macerated twice with diethyl ether. The resulting residue was dissolved in dichloromethane and washed with diethyl ether. The ethereal layer was decanted off. The solid thus obtained was dried under high vacuum to furnish title compound. Yield: 20 g.

### Step c: Synthesis of (1R)-7-bromo-2-(1′S-phenylethyl)-2-azabicyclo[2.2.1]heptane

To a solution of the compound obtained from step b above (19.9 g, 0.057 mol) in dry tetrahydrofuran at −10° C. was added sodium bis(2-methoxyethoxy) aluminium hydride (0.115 mol) and stirred the reaction mixture at −10° C. for 2 hours. The mixture was quenched with saturated sodium bicarbonate solution. The mixture was filtered over celite pad. The filtrate thus obtained was washed with water and brine, dried over anhydrous sodium sulphate and concentrated under reduced pressure. The residue thus obtained was purified by column chromatography using 1% ethyl acetate in hexane as eluent to furnish the title compound.

¹H NMR (CDCl₃): δ 7.32-7.21 (5H, m), 4.19 (1H, s), 3.54 (1H, m), 3.11 (1H, s), 3.02 (1H, bm), 2.38 (1H, s), 2.28 (2H, m), 1.9 (1H, m), 1.8 (2H, m), 1.25 (3H, m)

### Synthesis of 2,2-diphenylpropanoic Acid

The title compound is commercially available.

### Synthesis of hydroxy(diphenyl)acetic Acid

The title compound was prepared following the procedure as described in Vogel's Text Book of Practical Organic Chemistry, Page 1046 (5^{th} Ed).

### Synthesis of cyclopentyl(hydroxy)phenylacetic Acid

The title compound was prepared following the procedure as described in Syn. Comm., 11(12), 943-946 (1981).

### Synthesis of 2-benzyl-7-bromo-2-azabicyclo[2.2.1]heptane

The title compound was prepared following the procedure as described in U.S. Pat. No. 6,559,171.

### Synthesis of 2-benzyl-2-azabicyclo[2.2.1]heptan-7-amine

### Step I: Synthesis of 2-(2-benzyl-2-azabicyclo[2.2.1]hept-7-yl)1H-isoindole-1,3(2H)-dione

A solution of the compound 2-benzyl-7-bromo-2-azabicyclo[2.2.1]heptane (0.3 g, 1.27 mmol), potassium phthalimide (0.23 gm, 1.24 mmol) in dimethylformamide (10 ml) was stirred at 100° C. for 5-6 hours. The reaction mixture was poured into water and extracted with ethylacetate. The organic layer was washed with water and brine, dried over anhydrous sodium sulphate and concentrated under reduced pressure. The residue thus obtained was purified by column chromatography using 20% Ethyl acetate in hexane as eluent. Yield=0.2 g.

### Step II: 2 benzyl-2-azabicyclo[2.2.1]heptan-7-amine

To the solution of the compound obtained from step I above (0.17 g, 0.512 mmol) in ethanol (5 ml) was added hydrazine hydrate (0.024 ml, 0.512 mmol) and refluxed for 2 hours. To the resulting reaction mixture was added concentrated hydrochloric acid (2 ml) and the solution was again refluxed for one hour thirty minutes and then subsequently stirred at room temperature for overnight. The mixture was filtered and the filtrate was concentrated under reduced pressure. The residue thus obtained was basified with sodium hydroxide till the solution attained pH 12. The mixture was extracted with ethyl acetate, washed the organic layer with water and brine, dried over anhydrous sodium sulphate and concentrated under reduced pressure to furnish the title compound. Yield: 80 mg.

¹H NMR (CDCl₃): δ 7.34-7.20 (m, 5H), 3.69 (s, 2H), 3.44 (s, 1H), 3.01-2.98 (m, 2H), 2.91 (s, 1H), 2.23-2.21 (d, J=8H, 2H), 1.83 (bs, 1H), 1.47-1.37 (m, 2H).

### Example 1

### Synthesis of 2-benzyl-2-azabicyclo[2.2.1]hept-7-yl 2,2-diphenylpropanoate (Compound No. 11)

To the solution of 2,2-diphenylpropanoic acid (1.69 mmol) and 2-benzyl-7-bromo-2-azabicyclo[2.2.1]heptane (1.12 mmol) in dry toluene, was added 1,8-diazabicyclo[5.4.0]undec-7-ene (2.25 mmol) and refluxed overnight. The reaction mixture was then concentrated under vacuum. The residue thus obtained was purified by column chromatography using 8% ethyl acetate in hexane as eluent to furnish the title compound.

Yield: 230 mg.

¹H NMR (CDCl₃): δ 7.31-7.19 (15H, m), 5.08 (1H, s), 3.66 (2H, s), 3.13 (1H, s), 2.96-2.93 (1H, m), 2.30-2.25 (2H, m), 1.89 (3H, s), 1.32-1.23 (4H, m).

Mass (m/z): 412 (M⁺+1).

Analogues of 2-benzyl-2-azabicyclo[2.2.1]hept-7-yl 2,2-diphenylpropanoate (Compound No. 11 described below, were prepared similarly, by coupling racemic or optically active acid with racemic or optically active amine:
2-Benzyl-2-azabicyclo[2.2.1]hept-7-yl hydroxy(diphenyl)acetate (Compound No. 1)
2-Benzyl-2-azabicyclo[2.2.1]hept-7-yl cyclopentyl(hydroxy)-2-thienylacetate (Compound No. 2)
2-Benzyl-2-azabicyclo[2.2.1]hept-7-yl cyclopentyl(hydroxy)phenylacetate (Compound No 5)
2-Benzyl-2-azabicyclo[2.2.1]hept-7-yl hydroxy(phenyl)-2-thienylacetate (Compound No. 6)
2-Benzyl-2-azabicyclo[2.2.1]hept-7-yl hydroxy[bis(3-methylphenyl)]acetate (Compound No. 17)
2-Benzyl-2-azabicyclo[2.2.1]hept-7-yl methoxy(diphenyl)acetate (Compound No. 18)
2-[(1′R)-1-phenylethyl]-(1S or 1R)-2-azabicyclo[2.2.1]hept-7-yl (2R)-cyclopentyl(hydroxy)phenylacetate (Compound No. 30)
2-[(1′S)-1-phenylethyl]-(1R or 1S)-2-azabicyclo[2.2.1]hept-7-yl cyclohexyl(hydroxy)phenylacetate (Compound No. 32)
2-[(1′S)-1-phenylethyl]-(1R or 1S)-2-azabicyclo[2.2.1]hept-7-yl cyclopentyl(hydroxy)phenylacetate (Compound No. 33)
2-[(1′S)-1-phenylethyl]-(1R or 1S)-2-azabicyclo[2.2.1]hept-7-yl (2R)-[(1S or 1R) (3,3-difluorocyclopentyl)(hydroxy)phenylacetate (Compound No. 34) and 2-[(1′S)-1-phenylethyl]-(1R or 1S)-2-azabicyclo[2.2.1]hept-7-yl (2R)-[(1R or 1S) (3,3-difluorocyclopentyl)](hydroxy)phenylacetate (Compound No. 35)
   The title diastereomers were separated by column chromatography.
2-[(1′S)-1-phenylethyl]-(1R or 1S)-2-azabicyclo[2.2.1]hept-7-yl (2R)-cyclopentyl(hydroxy)phenylacetate (Compound No. 36)
2-[(1′S)-1-Phenylethyl]-(1R or 1S)-2-azabicyclo[2.2.1]hept-7-yl cycloheptyl(hydroxy)-2-thienylacetate (Compound No. 84)
2-[(1′S)-1-Phenylethyl]-(1R or 1S)-2-azabicyclo[2.2.1]hept-7-yl hydroxy(phenyl)-2-thienylacetate (Compound No. 85)
2-[(1′S)-1-Phenylethyl]-2-azabicyclo[2.2.1]hept-7-yl cycloheptyl(hydroxy)phenylacetate (Compound No. 86)
2-[(1′S)-1-Phenylethyl]-2-azabicyclo[2.2.1]hept-7-yl hydroxy(diphenyl)acetate (Compound No. 87)
2-[(1′S)-1-Phenylethyl]-(1R or 1S)-2-azabicyclo[2.2.1]hept-7-yl hydroxy(4-methylphenyl)phenylacetate (Compound No. 88)
2-[(1S)-1-Phenylethyl]-(1R or 1S)-2-azabicyclo[2.2.1]hept-7-yl (4-fluorophenyl)(hydroxy)phenylacetate (Compound No. 89)
2-[(1′S)-1-phenylethyl]-(1R or 1S)-2-azabicyclo[2.2.1]hept-7-yl cyclopentyl(phenyl)acetate (Compound No. 90)
2-[(1′S)-1-Phenylethyl]-(1R or 1S)-2-azabicyclo[2.2.1]hept-7-yl-(2R)-[(1S)-3,3-difluorocyclohexyl](hydroxy)phenylacetate (Compound No. 91)
2-[(1′S)-1-phenylethyl]-(1R or 1S)-2-azabicyclo[2.2.1]hept-7-yl (2R)-cyclopentyl(hydroxy)phenylacetate (Compound No. 92)
2-[(1′S)-1-Phenylethyl]-(1R or 1S)-2-azabicyclo[2.2.1]hept-7-yl (2R or 2S)-cycloheptyl(hydroxy)phenylacetate (Compound No. 93)
2-[(1′S)-1-Phenylethyl]-(1R or 1S)-2-azabicyclo[2.2.1]hept-7-yl cyclohexyl(hydroxy)phenylacetate (Compound No. 96)
2-[(1′S)-1-Phenylethyl]-(1R or 1S)-2-azabicyclo[2.2.1]hept-7-yl-(2R)-[(1S)-3,3-difluorocyclopentyl](hydroxy)phenylacetate (Compound No. 102)
2-[(1′S)-1-phenylethyl]-(1R or 1S)-2-azabicyclo[2.2.1]hept-7-yl-(2R)-[(1R)-3,3-difluorocyclopentyl](hydroxy)phenylacetate (Compound No. 103)
2-[(1′S)-1-phenylethyl]-(1R or 1S)-2-azabicyclo[2.2.1]hept-7-yl-(2R)-[(1R)-3,3-difluorocyclohexyl](hydroxy)phenylacetate (Compound No. 104)
2-[(1′R)-1-Phenylethyl]-(1S or 1R)-2-azabicyclo[2.2.1]hept-7-yl cycloheptyl(hydroxy)phenylacetate (Compound No. 105)

### Example 2

### Synthesis N-(2-benzyl-2-azabicyclo[2.2.1]hept-7-yl)-2,2-diphenylpropanamide (Compound No. 7)

To the solution of 2,2-diphenylpropanoic acid (1.32 mmol) and 3-azabicyclo[3.2.1]octan-8-amine (1.46 mmol) in dimethylformamide cooled in ice bath, was added N-methyl morpholine (2.63 mmol) and 1-hydroxybenzotriazole (1.46 mmol). The reaction mixture was stirred at the 0° C. for one hour followed by the addition of 1-(3-dimethylaminopropyl)-3-ethylcarbodiimide hydrochloride (1.32 mmol) and the resulting reaction mixture was stirred at same temperature for one hour and then left at room temperature for overnight. The reaction mixture was quenched by addition of water and extracted with ethylacetate. The organic layer was separated, washed with water and brine, dried over sodium sulphate and concentrated under reduced pressure. The compound was then purified by column chromatography using 25% ethylacetate in hexane as eluent to furnish the title compound. Yield: 180 mg.

¹H NMR (CDCl₃): δ 7.33-7.19 (15H, m), 5.32-5.30 (1H, m), 4.23-4.21 (1H, m), 3.74-3.68 (2H, m), 3.10 (1H, s), 2.90-2.88 (1H, m), 2.23-1.99 (6H, m), 1.11-0.98 (3H, m).

Mass (m/z): 411 (M⁺+1).

IR (DCM): 1669.

Analogues of N-(2-benzyl-2-azabicyclo[2.2.1]hept-7-yl)-2,2-diphenylpropanamide (Compound No. 7) described below were prepared similarly,
N-(2-benzyl-2-azabicyclo[2.2.1]hept-7-yl)-2-cyclopentyl-2-hydroxy-2-(2-thienyl)acetamide (Compound No. 8)
N-(2-benzyl-2-azabicyclo[2.2.1]hept-7-yl)-2-hydroxy-2-phenyl-2-(2-thienyl)acetamide (Compound No. 9)
N-(2-benzyl-2-azabicyclo[2.2.1]hept-7-yl)-2-cyclopentyl-2-hydroxy-2-phenylacetamide (Compound No. 10)
N-(2-benzyl-2-azabicyclo[2.2.1]hept-7-yl)-2-hydroxy-2,2-diphenylacetamide (Compound No. 13)

### Example 3

### Synthesis of N-2-azabicyclo[2.2.1]hept-7-yl-2,2-diphenylpropanamide (Compound No. 15)

To the solution of the Compound No. 7 (125 mg. 0.30 mmol) in methanol, was added palladium on carbon (10% w/w) and ammonium formate (1.76 mmol). The reaction mixture was refluxed for 3 hours. The mixture was filtered through celite pad and washed with methanol. The filtrate was concentrated under vacuum. The crude compound was diluted with water and acidified using concentrated hydrochloric acid. Impurities were extracted with dichloromethane. The aqueous layer was basified and extracted with ethyl acetate. The organic layer was separated, washed with water and brine, dried over anhydrous sodium sulphate and concentrated under reduced pressure to furnish the title compound.

¹H NMR (CDCl₃): δ 7.36-7.20 (10H, m), 5.35-5.30 (1H, m), 4.10-4.09 (1H, m), 3.62-2.37 (5H, m), 2.01-1.98 (3H, s), 1.29-0.86 (4H, m).

Analogues of N-2-azabicyclo[2.2.1]hept-7-yl-2,2-diphenylpropanamide (Compound No. 15) described below were prepared similarly,
2-Azabicyclo[2.2.1]hept-7-yl hydroxy(diphenyl)acetate (Compound No. 3)
2-Azabicyclo[2.2.1]hept-7-yl cyclopentyl(hydroxy)phenylacetate (Compound No. 4)
2-Azabicyclo[2.2.1]hept-7-yl 2,2-diphenylpropanoate (Compound No. 12)
N-2-Azabicyclo[2.2.1]hept-7-yl-2-hydroxy-2,2-diphenylacetamide (Compound No. 14)
N-2-Azabicyclo[2.2.1]hept-7-yl-2-cyclopentyl-2-hydroxy-2-phenylacetamide (Compound No. 16)
2-Azabicyclo[2.2.1]hept-7-yl (2R)-((1R or 1S)-3,3-difluorocyclopentyl)(hydroxy)phenylacetate (Compound No. 19)
2-(Pyridin-3-ylmethyl)-(1R or 1S)-2-azabicyclo[2.2.1]hept-7-yl cyclopentyl(hydroxy)phenylacetate (Compound No. 21),
(1S or 1R)-2-Azabicyclo[2.2.1]hept-7-yl (2R)-cyclopentyl(hydroxy)phenylacetate (Compound No. 31)
(1R or 1S)-2-Azabicyclo[2.2.1]hept-7-yl (2R)-cyclopentyl(hydroxy)phenylacetate (Compound No. 37)
(1R or 1S)-2-Azabicyclo[2.2.1]hept-7-yl cyclopentyl(hydroxy)phenylacetate (Compound No. 38)

### Example 4

### Synthesis of 2-(pyridin-3-ylmethyl)-(1R)-2-azabicyclo[2.2.1]hept-7-yl cyclopentyl(hydroxy)phenylacetate (Compound No. 21)

Pyridine 3-carboxaldehyde (0.342 mmol) was added to a solution of Compound No. 38 (0.342 mmol) in tetrahydrofuran (5 ml) followed by addition of sodium triacetoxyborohydride (1.19 mmol) and acetic acid (0.462 mmol). The reaction mixture was stirred at room temperature for overnight. The mixture was poured into saturated sodium bicarbonate solution. The organic layer was separated and the aqueous layer was extracted with ethylacetate. The combined organic layers were washed with brine, dried over anhydrous sodium sulphate and concentrated under reduced pressure. The residue thus obtained was purified by column chromatography to furnish the title compound.

H¹NMR (CDCl₃) δ: 8.52-8.46 (2H, m), 7.67-7.58 (3H, m), 7.32-7.19 (3H, m), 5.29 (1H, s), 3.69-3.66 (3H, d), 3.23-3.09 (1H, d), 2.92 (2H, bs), 2.32-2.24 (4H, m), 1.80-1.25 (11H, m).

Following compounds were prepared similarly,
2-(1,3-Benzodioxol-5-ylmethyl)-(1R or 1S)-2-azabicyclo[2.2.1]hept-7-yl cyclopentyl(hydroxy)phenylacetate (Compound No. 22)
2-(Cyclohexylmethyl)-(1R)-2-azabicyclo[2.2.1]hept-7-yl cyclopentyl(hydroxy)phenylacetate (Compound No. 23)
2-(4-Methylbenzyl)-(1R or 1S)-2-azabicyclo[2.2.1]hept-7-yl cyclopentyl(hydroxy)phenylacetate (Compound No. 24)
2-(2-Fluorobenzyl)-(1R or 1S)-2-azabicyclo[2.2.1]hept-7-yl cyclopentyl(hydroxy)phenylacetate (Compound No. 25)
2-(4-Fluorobenzyl)-(1R or 1S)-2-azabicyclo[2.2.1]hept-7-yl cyclopentyl(hydroxy)phenylacetate (Compound No. 26)
2-(3-Methoxybenzyl)-(1R or 1S)-2-azabicyclo[2.2.1]hept-7-yl cyclopentyl(hydroxy)phenylacetate (Compound No. 27)
2-(3-Methylbenzyl)-(1R or 1S)-2-azabicyclo[2.2.1]hept-7-yl cyclopentyl(hydroxy)phenylacetate (Compound No. 29)

### Example 5

### Synthesis of 2-methyl-(1R or 1S)-2-azabicyclo[2.2.1]hept-7-yl cyclopentyl(hydroxy)phenylacetate (Compound No. 28)

To a solution of Compound No. 38 (0.2 g, 0.685 mmol) in acetonitrile (3 ml) and formaldehyde (2 ml), was added sodium cyanoborohydride (0.21 g, 34.24 mmol) at 25-30° C. The reaction mixture was stirred overnight at room temperature and subsequently neutralized with acetic acid. The reaction mixture was again stirred for 30 minutes at the same temperature. The solvent was removed under reduced pressure and the residue thus obtained was diluted with water and basified to pH=14 with sodium hydroxide (10%). The reaction mixture was extracted with ethyl acetate, dried over anhydrous sodium sulphate and concentrated under reduced pressure. The residue thus obtained was purified by column chromatography using 5% methanol in dichloromethane and 1% ammonia solution to furnish the title compound. Yield=140 g.

¹H NMR (CDCl₃) δ: 7.62-7.60 (m, 2H), 7.34-7-24 (m, 3H), 4.972 (s, 1H), 3.16-3.01 (d, 1H), 2.91 (m, 2H), 2.35-2.31 (m, 5H), 1.56-1.30 (m, 13H).

Following compounds were prepared similarly,
(1R or 1S)-2-Methyl-2-azabicyclo[2.2.1]hept-7-yl-(2R)-cyclopentyl(hydroxy)phenylacetate (Compound No. 81)
(1S or 1R)-2-Methyl-2-azabicyclo[2.2.1]hept-7-yl-(2R)-cyclopentyl(hydroxy)phenylacetate (Compound No. 82)
(1R or 1S)-2-Methyl-2-azabicyclo[2.2.1]hept-7-yl cyclopentyl(hydroxy)phenylacetate (Compound No. 83)
(1R or 1S)-2-Methyl-2-azabicyclo[2.2.1]hept-7-yl cycloheptyl(hydroxy)phenylacetate (Compound No. 94)
(1R or 1S)-2-Methyl-2-azabicyclo[2.2.1]hept-7-yl (2R)-[(1R)-3,3-difluorocyclopentyl](hydroxy)phenylacetate (Compound No. 95)
(1R or 1S)-2-Methyl-2-azabicyclo[2.2.1]hept-7-yl (2R)-[(1S)-3,3-difluorocyclohexyl](hydroxy)phenylacetate (Compound No. 97)
(1R or 1S)-2-Methyl-2-azabicyclo[2.2.1]hept-7-yl 2,2-diphenylpropanoate (Compound No. 98)
(1R or 1S)-2-Methyl-2-azabicyclo[2.2.1]hept-7-yl (2R)-[(1R)-3,3-difluorocyclohexyl](hydroxy)phenylacetate (Compound No. 99)
(1R or 1S)-2-Methyl-2-azabicyclo[2.2.1]hept-7-yl (2R)-[(1R)-3,3-difluorocyclopentyl](hydroxy)phenylacetate (Compound No. 100)
(1R or 1S)-2-Methyl-2-azabicyclo[2.2.1]hept-7-yl cycloheptyl(hydroxy)phenylacetate (Compound No. 101)

### Example 6

### Synthesis of 7-{[cyclopentyl(hydroxy)phenylacetyl]oxy}-(1R or 1S)-2-(3-methoxybenzyl)-2-methyl-2-azoniabicyclo[2.2.1]heptane Iodide (Compound No. 40)

To a solution of the 2-(3-methoxybenzyl)-(1R)-2-azabicyclo[2.2.1]hept-7-yl cyclopentyl(hydroxy)phenylacetate (40 mg) in dichloromethane (0.5 ml) was added methyl iodide (excess) and stirred the reaction mixture at room temperature overnight. The reaction mixture was concentrated under reduced pressure followed by the addition of diethyl ether. The precipitates thus formed were washed with diethyl ether. Supernatant was decanted off and the precipitates were died under reduced pressure to furnish the title compound. Yield: 25 mg

¹H NMR (CD₃OH): δ 7.58 (2H, m), 7.30-7.16 (6H, m), 7.0 (1H, bs), 5.7-5.5 (1H, m), 4.95-4.81 (2H, m), 4.4-3.9 (2H, m), 3.74-3.70 (4H, m), 3.19 (2H, d, 6.68 Hz), 2.91 (1H, m), 2.7 (2H, bs), 1.8-1.1 (13H, m).

Mass spectrum (m/z, +ve ion mode): 450 (M⁺+1).

Following compounds were prepared similarity,
(1R or 1S)-7-{[(2R)-2-cyclopentyl-2-hydroxy-2-phenylacetyl]oxy}-2,2-dimethyl-2-azoniabicyclo[2.2.1]heptane Iodide (Compound No. 39)
7-{[cyclopentyl(hydroxy)phenylacetyl]oxy}-(1R or 1S)-2-(4-fluorobenzyl)-2-methyl-2-azoniabicyclo[2.2.1]heptane Iodide (Compound No. 41)
7-{[cyclopentyl(hydroxy)phenylacetyl]oxy}-(1R or 1S)-2-(2-fluorobenzyl)-2-methyl-2-azoniabicyclo[2.2.1]heptane Iodide (Compound No. 42)
7-{[cyclopentyl(hydroxy)phenylacetyl]oxy}-(1R or 1S)-2-methyl-2-(4-methylbenzyl)-2-azoniabicyclo[2.2.1]heptane Iodide (Compound No. 43)
2-(cyclohexylmethyl)-7-{[cyclopentyl(hydroxy)phenylacetyl]oxy}-(1R or 1S)-2-methyl-2-azoniabicyclo[2.2.1]heptane Iodide (Compound No. 44)
7-{[(2R)-2-cyclopentyl-2-hydroxy-2-phenylacetyl]oxy}-(1S or 1R)-2,2-dimethyl-2-azoniabicyclo[2.2.1]heptane Iodide (Compound No. 45)
7-[2-cycloheptyl(hydroxy)-2-thienylacetoxy]-2-methyl-2-[(1′S)-1-phenylethyl]-(1R or 1S)-2-azoniabicyclo[2.2.1]heptane Iodide (Compound No. 46)
7-{[hydroxy(phenyl)-2-thienylacetyl]oxy}-2-methyl-2-[(1′S)-1-phenylethyl]-(1R or 1S)-2-azoniabicyclo[2.2.1]heptane Iodide (Compound No. 47)
7-[2-cycloheptyl(hydroxy)phenylacetoxy]-2-methyl-2-[(1′S)-1-phenylethyl]-(1R or 1S)-2-azoniabicyclo[2.2.1]heptane Iodide (Compound No. 48)
7-[2-hydroxy(diphenyl)acetoxy]-2-methyl-2-[(1′S)-1-phenylethyl]-(1R or 1S)-2-azoniabicyclo[2.2.1]heptane Iodide (Compound No. 49)
7-[2-hydroxy(4-methylphenyl)phenylacetoxy]-2-methyl-2-[(1′S)-1-phenylethyl]-(1R or 1S)-2-azoniabicyclo[2.2.1]heptane Iodide (Compound No. 50)
7-[2-(4-fluorophenyl)(hydroxy)phenylacetoxy]-2-methyl-2-[(1′S)-1-phenylethyl]-(1R or 1S)-2-azoniabicyclo[2.2.1]heptane Iodide (Compound No. 51)
7-{[cyclopentyl(phenyl)acetyl]oxy}-2-methyl-2-[(1′S)-1-phenylethyl]-(1R or 1S)-2-azoniabicyclo[2.2.1]heptane Iodide (Compound No. 52)
7-({(2R)-2-[(1S)-3,3-difluorocyclohexyl]-2-hydroxy-2-phenylacetyl}oxy)-2-methyl-2-[(1′S)-1-phenylethyl]-(1R or 1S)-2-azoniabicyclo[2.2.1]heptane iodide (Compound No. 53)
7-{[(2R)-2-cyclopentyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 54)
7-{[(2R or 2S)-2-cycloheptyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 55)
2-(1,2,3-benzotrioxol-5-ylmethyl)-7-[(2-cycloheptyl-2-hydroxy-2-phenylacetyl)oxy]-(1R or 1S)-2-methyl-2-azoniabicyclo[2.2.1]heptane Bromide (Compound No. 56)
7-[2-cycloheptyl(hydroxy)phenylacetoxy]-2-ethyl-(1R or 1S)-2-methyl-2-azoniabicyclo[2.2.1]heptane Iodide (Compound No. 57)
7-[2-cycloheptyl(hydroxy)phenylacetoxy]-(1R or 1S)-2-methyl-2-propyl-2-azoniabicyclo[2.2.1]heptane Iodide (Compound No. 58)
7-[2-cycloheptyl(hydroxy)phenylacetoxy]-(1R or 1S)-2-(4-fluorobenzyl)-2-methyl-2-azoniabicyclo[2.2.1]heptane Bromide (Compound No. 59)
7-[2-cycloheptyl(hydroxy)phenylacetoxy]-2-(4-methoxybenzyl)-(1R or 1S)-2-methyl-2-azoniabicyclo[2.2.1]heptane Chloride (Compound No. 60)
7-{[cycloheptyl(hydroxy)phenylacetyl]oxy}-(1R or 1S)-2-methyl-2-(2-methylprop-2-en-1-yl)-2-azoniabicyclo[2.2.1]heptane Bromide (Compound No. 61)
2-benzyl-7-[2-cycloheptyl(hydroxy)phenylacetoxy]-(1R or 1S)-2-methyl-2-azoniabicyclo[2.2.1]heptane Bromide (Compound No. 62)
7-[2-cycloheptyl(hydroxy)phenylacetoxy]-(1R or 1S)-2-methyl-2-(3-phenylpropyl)-2-azoniabicyclo[2.2.1]heptane Bromide (Compound No. 63)
7-({(2R)-2-[(1R)-3,3-difluorocyclopentyl]-2-hydroxy-2-phenylacetyl}oxy)-(1R or 1S)-2-methyl-2-propyl-2-azoniabicyclo[2.2.1]heptane Bromide (Compound No. 64)
7-({(2R)-2-[(1R)-3,3-difluorocyclopentyl]-2-hydroxy-2-phenylacetyl}oxy)-(1R or 1S)-2-methyl-2-(3-phenylpropyl)-2-azoniabicyclo[2.2.1]heptane Bromide (Compound No. 65)
7-({(2R)-2-[(1R)-3,3-difluorocyclopentyl]-2-hydroxy-2-phenylacetyl}oxy)-(1R or 1S)-2-(4-fluorobenzyl)-2-methyl-2-azoniabicyclo[2.2.1]heptane Bromide (Compound No. 66)
7-({(2R)-2-[(1R)-3,3-difluorocyclopentyl]-2-hydroxy-2-phenylacetyl}oxy)-(1R or 1S)-2-methyl-2-(2-methylprop-2-en-1-yl)-2-azoniabicyclo[2.2.1]heptane Bromide (Compound No. 67)
2-(1,3-benzodioxol-5-ylmethyl)-7-({(2R)-2-[(1R)-3,3-difluorocyclopentyl]-2-hydroxy-2-phenylacetyl}oxy)-(1R or 1S)-2-methyl-2-azoniabicyclo[2.2.1]heptane Bromide (Compound No. 68)
7-({(2R)-2-[(1R)-3,3-difluorocyclopentyl]-2-hydroxy-2-phenylacetyl}oxy)-(1R or 1S)-2-(4-methoxybenzyl)-2-methyl-2-azoniabicyclo[2.2.1]heptane Bromide (Compound No. 69)
7-{[(2R)-2-cycloheptyl-2-hydroxy-2-phenylacetyl]oxy}-(1R or 1S)-2,2-dimethyl-2-azoniabicyclo[2.2.1]heptane Iodide (Compound No. 70)
7-[2-cyclohexyl(hydroxy)phenylacetoxy]-2-methyl-2-[(1S)-1-phenylethyl]-(1R or 1S)-2-azoniabicyclo[2.2.1]heptane Iodide (Compound No. 71)
7-({(2R)-2-[(1S)-3,3-difluorocycloheptyl]-2-hydroxy-2-phenylacetyl}oxy)-(1R or 1S)-2,2-dimethyl-2-azoniabicyclo[2.2.1]heptane Iodide (Compound No. 72)
7-[(2,2-diphenylpropanoyl)oxy]-(1R or 1S)-2,2-dimethyl-2-azoniabicyclo[2.2.1]heptane Iodide (Compound No. 73)
7-({(2R)-2-[(1R)-3,3-difluorocyclohexyl]-2-hydroxy-2-phenylacetyl}oxy)-(1R or 1S)-2,2-dimethyl-2-azoniabicyclo[2.2.1]heptane Iodide (Compound No. 74)
7-[2-cycloheptyl(hydroxy)phenylacetoxy]-2-methyl-2-[(1′R)-1-phenylethyl]-(1S or 1R)-2-azoniabicyclo[2.2.1]heptane Iodide (Compound No. 75)
7-({(2R)-2-[(1R)-3,3-difluorocyclopentyl]-2-hydroxy-2-phenylacetyl}oxy)-(1R or 1S)-2,2-dimethyl-2-azoniabicyclo[2.2.1]heptane Iodide (Compound No. 76)
7-[2-cycloheptyl(hydroxy)phenylacetoxy]-(1S or 1R)-2,2-dimethyl-2-azoniabicyclo[2.2.1]heptane Iodide (Compound No. 77)
7-{(2R)-2-[(1S)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 78)
7-{(2R)-2-[(1R)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 79)
7-{(2R)-[(1R)-3,3-difluorocyclohexyl](hydroxy)phenylacetoxy}-2-methyl-2-[(1′S)-1-phenylethyl]-(1R or 1S)-2-azoniabicyclo[2.2.1]heptane iodide (Compound No. 80)

### Biological Activity

### Radioligand Binding Assays:

The affinity of test compounds for M₂ and M₃ muscarinic receptor subtypes was determined by [³H]-N-methylscopolamine binding studies using rat heart and submandibular gland respectively as described by Moriya et al., (Life Sci, 1999, 64(25): 2351-2358) with minor modifications. In competition binding studies, specific binding of [3H] NMS was also determined using membranes from Chinese hamster ovary (CHO) cells expressing cloned human M₁, M₂, M₃, M₄ and M₅ receptors. Selectivities were calculated from the Ki values obtained on these human cloned membranes.

### Membrane Preparation:

### (a) Rat Tissues

Submandibular glands and heart were isolated and placed in ice-cold homogenising buffer (HEPES 20 mM, 10 mM EDTA, pH 7.4) immediately after sacrifice. The tissues were homogenised in ten volumes of homogenising buffer and the homogenate was filtered through two layers of wet gauze and filtrate was centrifuged at 500 g for 10 min. The supernatant was subsequently centrifuged at 40,000 g for 20 min. The pellet thus obtained was resuspended in assay buffer (HEPES 20 mM, EDTA 5 mM, pH 7.4) and were stored at −70° C. until the time of assay.

### (b) CHO Cells Expressing Human Recombinant Receptors

The cell pellets were homogenised for 30 sec at 12,000 to 14,000 rpm, with intermittent gaps of 10-15 sec in ice-cold homogenising buffer (20 mM HEPES, 10 mM EDTA, pH 7.4). The homogenate was then centrifuged at 40,000 g for 20 min at 4° C. The pellet thus obtained was resuspended in homogenising buffer and was stored at −70° C. until the time of assay.

Ligand binding assay: The compounds were dissolved and diluted in DMSO. The membrane homogenates (150-250 μg protein) were incubated in 250 μl of assay volume (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice-cold 50 mM Tris HCl buffer (pH 7.4). The filter mats were dried and bound radioactivity retained on filters was counted. The IC₅₀ & K_{d} were estimated by using the non-linear curve fitting program using G Pad Prism software. The value of inhibition constant Kᵢ was calculated from competitive binding studies by using Cheng & Prusoff equation (Biochem Pharmacol, 1973, 22: 3099-3108), Ki=IC₅₀/(1+L/Kd), where L is the concentration of [³H]NMS used in the particular experiment. pki is −log [Ki].

The above disclosed compounds (compounds 1-18) showed Ki values for rat M₂ and M₃ receptors in the range of from about 2 nM to 20 nM, or from about 50 nM to 500 nM, or more than 500 nM.
The above disclosed compounds (compounds 2-6, 12, 19-32, 34-37 and 39-82) showed Ki values for human M₂ and M₃ receptors in the range of from about 0.04 nM to 0.4 nM, or from about 4 nM to 40 nM, or from about 40 nM to 550 nM.

### Functional Experiments Using Isolated Rat Bladder:

### Methodology:

Animals are euthanized by overdose of thiopentone and whole bladder is isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder is cut into longitudinal strips (3 mm wide and 5-6 mm long) and mounted in 10 ml organ baths at 30° C., with one end connected to the base of the tissue holder and the other end connected through a force displacement transducer. Each tissue is maintained at a constant basal tension of 1 g and allowed to equilibrate for 1½ hour during which the Tyrode buffer is changed every 15-20 min. At the end of equilibration period the stabilization of the tissue contractile response is assessed with 1 μmol/L of Carbachol till a reproducible response is obtained. Subsequently a cumulative concentration response curve to carbachol (10⁻⁹ mol/L to 3×10⁻⁴ mol/L) is obtained. After several washes, once the baseline is achieved, cumulative concentration response curve is obtained in presence of NCE (NCE added 20 min. prior to the second cumulative response curve.

The contractile results are expressed as % of control E max. ED50 values are calculated by fitting a non-linear regression curve (Graph Pad Prism). pKb values are calculated by the formula pKb=−log [(molar concentration of antagonist/(dose ratio−1))]

where,
dose ratio=ED50 in the presence of antagonist/ED50 in the absence of antagonist.

### In-Vitro Functional Assay to Evaluate Efficacy of MRA on Guinea Pig Trachea

### Animals and Anesthesia

The Guinea Pigs (300-900 gm) were procured from experimental animal facility at Ranbaxy Research laboratories. The trachea was removed under an overdose of anesthesia (sodium pentobarbital, ˜300 mg/kg i.p) and immediately was kept it in ice-cold Krebs Henseleit buffer of the following composition (mM): NaCl, 118; KCl 4.7; CaCl₂, 2.5; MgSO₄, 1.2; NaHCO₃, 25; KH₂PO₄, 1.2, glucose 11.1.

### Trachea Experiments:

The tissue of adherent fascia was cleaned and was cut it into seven-eight strips of equal size (with approx. 4-5 tracheal rings in each strip). The trachea was cut along the mid-dorsal surface with the smooth muscle band intact and made a series of transverse cuts from alternate sides so that they do not transect the preparation completely. Opposite end of the cut rings were tied with the help of a thread. The tissue was mounted in isolated tissue baths containing 10 ml Krebs Henseleit buffer maintained at 37° C. and bubbled with carbogen (95% oxygen and 5% carbon dioxide), at a basal tension of 1 gm. The buffer was changed 3-4 times for about an hour. The tissues were equilibrated for 1 hr for stabilization. After 1 hr, the tissue was challenged with 60 mM KCl. This was repeated after every 2-3 washes till two similar consecutive responses were obtained. At the end of stabilization, performed a carbachol concentration-response curve on all the tissues. The tissues were washed till the baseline was obtained. Thereafter, each tissue was incubated with different concentrations of MRA/Standard/Vehicle for 20 minutes followed by a second cumulative dose response curve to carbachol. The contractile response of tissues was recorded either on Powerlab system or on Grass polygraph (Model 7). Responses to carbachol were standardized as a percentage of the maximum carbachol response of the control CRC. Determined the carbachol EC₅₀ values in the presence and absence of inhibitor using graph pad prism. The pK_{B} value was calculated, as an index of functional antagonism from EC₅₀ data using the following relationship:

−log [antagonist (M)/(EC₅₀ antagonist/EC₅₀ control)−1]

The data was expressed as mean±s.e.m for n observations. In tissues where Eₘₐₓ attained was less than 50% pK_{B} was calculated by Kenakin's double reciprocal plot.

All drugs and chemicals used in the study were of AR grade. Carbachol was procured from Sigma Chemicals, U.S.A. Stock solutions of Standard/NCEs were prepared in DMSO. Subsequent dilutions were prepared from the stock in MilliQ water

The pKb values of two of the compounds (Compounds 48 and 76) were in the range from about 8 nM to 11 nM.

In-Vitro Functional Assay to Evaluate Efficacy of “MRA” in Combination with “PDE-IV Inhibitors”

### Animals and Anesthesia:

Guinea Pig (400-600 gm) is procured and trachea is removed under anesthesia (sodium pentobarbital, 300 mg/kg i.p) and is immediately kept in ice-cold Krebs Henseleit buffer. Indomethacin (10 uM) is present throughout the KH buffer to prevent the formation of bronchoactive prostanoids.

### Trachea Experiments:

The tissue of adherent fascia is cleaned and cut into strips of equal size (with approx. 4-5 tracheal rings in each strip). The epithelium is removed by careful rubbing, minimizing damage to the smooth muscle. The trachea is opened along the mid-dorsal surface with the smooth muscle band intact and a series of transverse cuts is made from alternate sides so that they do not transect the preparation completely. The opposite end of the cut rings is tied with the help of a thread. The tissue is mounted in isolated tissue baths containing 10 ml Krebs Henseleit buffer maintained at 37° C. and bubbled with carbogen, at a basal tension of 1 gm. The buffer is changed 4-5 times for about an hour. The tissue is equilibrated for 1 hr for stabilization. After 1 hr, the tissue is challenged with 1 uM carbachol. This is repeated after every 2-3 washes till two similar consecutive responses are obtained. At the end of stabilization, the tissues are washed for 30 minutes followed by incubation with suboptimal dose of MRA/Vehicle for 20 minutes prior to contraction of the tissues with 1 μM carbachol and subsequently the relaxant activity of the PDE-IV inhibitor [10⁻⁹ M to 10⁻⁴ M] is assessed on the stabilized developed tension/response. The contractile response of tissues is recorded either on Powerlab data acquisition system or on Grass polygraph (Model 7). The relaxation is expressed as percentage of maximum carbachol response. The data is expressed as mean±s.e. mean for n observations. The EC₅₀ is calculated as the concentration producing 50% of the maximum relaxation to 1 μM carbachol. The percent relaxation is compared between the treated and control tissues using non-parametric unpaired t-test. A p value of <0.05 is considered to be statistically significant.

### In-Vivo Experiments

### In-Vivo Assay to Evaluate Efficacy of MRA Inhibitors

Male Guinea pigs are anesthetized with urethane (1.5 g/kg, i.p.). Trachea is cannulated along with jugular vein (for carbachol challenge) and animals are placed in the Plethysmograph-Box (PLY 3114 model; Buxco Electronics, Sharon, USA). Respiratory parameters are recorded using Pulmonary Mechanics Analyser, Biosystems XA software (Buxco Electronics, USA), which calculated lung resistance (R_{L}) on a breath-by-breath basis. Bronchoconstriction is induced by injections of Carbachol (10 μg/kg) delivered into the jugular vein. Increase in R_{L} over a period of 5 min post carbachol challenge is recorded in presence or absence of MRA or vehicle at 2 hrs and 12 hrs post treatment and expressed as % increase in R_{L} from basal

$$\% Inhibition = \frac{{R}_{L vehicle} - {R}_{L test}}{{R}_{L vehicle}} \times 100$$

R_{L vehicle} % increase in lung resistance from basal in vehicle treated
R_{L test} % increase in lung resistance from basal at a given dose of test
In-Vivo Assay to Evaluate Efficacy of MRA in Combination with PDE-IV Inhibitors

### Drug Treatment:

MRA (1 μg/kg to 1 mg/kg) and PDE-IV inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/ml) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchioalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS.
The percent inhibition data is used to compute ED₅₀ vales using Graph Pad Prism software (Graphpad Software Inc., USA).
3. In-Vivo Assay to Evaluate Efficacy of MRA in Combination with Corticosteroids

### Ovalbumin Induced Airway Inflammation:

Guinea pigs are sensitised on days 0, 7 and 14 with 50-μg ovalbumin and 10 mg aluminium hydroxide injected intraperitoneally. On days 19 and 20 guinea pigs are exposed to 0.1% w v⁻¹ ovalbumin or PBS for 10 min, and with 1% ovalbumin for 30 min on day 21. Guinea pigs are treated with test compound (0.1, 0.3 and 1 mg kg⁻¹) or standard 1 mg kg-1 or vehicle once daily from day 19 and continued for 4 days. Ovalbumin/PBS challenge is performed 2 hours after different drug treatment.

Twenty-four hours after the final ovalbumin challenge BAL is performed using Hank's balanced salt solution (HBSS). Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and eosinophil count are expressed as cell count (millions cells ml⁻¹ of BAL). Eosinophil is also expressed as percent of total leukocyte count. % inhibition is computed using the following formula.

$$\% Inhibition = \frac{{Eos}_{OVA} - {Eos}_{TEST}}{{Eos}_{OVA} - {Eos}_{CON}} \times 100$$

### Where,

Eos_{OVA}=Percentage of eosinophil in untreated ovalbumin challenged group
Eos_{TEST}=Percentage of eosinophil in group treated with a given dose of test compound
Eos_{CON}=Percentage of eosinophil in group not challenged with ovalbumin.
In-Vivo Assay to Evaluate Efficacy of “MRA” in Combination with p38 MAP Kinase Inhibitors
Lipopolysaccharide (LPS) induced airway hyperreactivity (AHR) and neutrophilia:

### Drug Treatment:

MRA (1 μg/kg to 1 mg/kg) and p38 MAP kinase inhibitor (1 μg/kg to 1 mg/kg) are instilled intratracheally under anesthesia either alone or in combination.

### Method:

Male wistar rats weighing 200±20 gm are used in the study. Rats have free access to food and water. On the day of experiment, animals are exposed to lipopolysaccharide (LPS, 100 μg/ml) for 40 min. One group of vehicle treated rats is exposed to phosphate buffered saline (PBS) for 40 min. Two hours after LPS/PBS exposure, animals are placed inside a whole body plethysmograph (Buxco Electronics, USA) and exposed to PBS or increasing acetylcholine (1, 6, 12, 24, 48 and 96 mg/ml) aerosol until Penh values (index of airway resistance) of rats attained 2 times the value (PC-100) seen with PBS alone. The respiratory parameters are recorded online using Biosystem XA software, (Buxco Electronics, USA). Penh, at any chosen dose of acetylcholine is, expressed as percent of PBS response and the using a nonlinear regression analysis PC100 (2 folds of PBS value) values are computed. Percent inhibition is computed using the following formula.

$$\% Inhibition = \frac{PC {100}_{LPS} - PC {100}_{TEST}}{PC {100}_{LPS} - PC {100}_{PBS}} \times 100$$

### Where,

PC100_{LPS}=PC100 in untreated LPS challenged group
PC100_{TEST}=PC100 in group treated with a given dose of test compound
PC100_{PBS}=PC100 in group challenged with PBS

Immediately after the airway hyperreactivity response is recorded, animals are sacrificed and bronchioalveolar lavage (BAL) is performed. Collected lavage fluid is centrifuged at 3000 rpm for 5 min, at 4° C. Pellet is collected and resuspended in 1 ml HBSS. Total leukocyte count is performed in the resuspended sample. A portion of suspension is cytocentrifuged and stained with Leishmann's stain for differential leukocyte count. Total leukocyte and Neutrophil counts are expressed as cell count (millions cells ml⁻¹ of BAL). Percent inhibition is computed using the following formula.

$$\% Inhibition = \frac{{NC}_{LPS} - {NC}_{TEST}}{{NC}_{LPS} - {NC}_{CON}} \times 100$$

### Where,

NC_{LPS}=Percentage of neutrophil in untreated LPS challenged group
NC_{TEST}=Percentage of neutrophil in group treated with a given dose of test compound
NC_{CON}=Percentage of neutrophil in group not challenged with LPS
The percent inhibition data is used to compute ED₅₀ vales using Graph Pad Prism software (Graphpad Software Inc., USA).
In-Vivo Assay to Evaluate Efficacy of “MRA” in Combination with β-Agonists

### Drug Treatment:

MRA (1 μg/kg to 1 mg/kg) and long acting β₂ agonist are instilled intratracheally under anesthesia either alone or in combination.

### Method

Wistar rats (250-350 gm) or balb/C mice (20-30 gm) are placed in body box of a whole body plethysmograph (Buxco Electronics, USA) to induce bronchoconstriction. Animals are allowed to acclimatise in the body box and are given successive challenges, each of 2 min duration, with PBS (vehicle for acetylcholine) or acetylcholine (i.e. 24, 48, 96, 144, 384, and 768 mg/ml). The respiratory parameters are recorded online using Biosystem XA software, (Buxco Electronics, USA) for 3 min. A gap of 2 min is allowed for the animals to recover and then challenged with the next higher dose of acetylcholine (ACh). This step is repeated until Penh of rats attained 2 times the value (PC-100) seen with PBS challenge. Following PBS/ACh challenge, Penh values (index of airway resistance) in each rat/mice is obtained in the presence of PBS and different doses of ACh. Penh, at any chosen dose of ACh is, expressed as percent of PBS response. The Penh values thus calculated are fed into Graph Pad Prism software (Graphpad Software Inc., USA) and using a nonlinear regression analysis PC100 (2 folds of PBS value) values are computed. % inhibition is computed using the following formula:

$$\% Inhibition = \frac{PC {100}_{TEST} - PC {100}_{CON}}{768 - PC {100}_{CON}} \times 100$$

### Where,

PC100_{CON}=PC100 in vehicle treated group
PC100_{TEST}=PC100 in group treated with a given dose of test compound
768=is the maximum amount of acetylcholine used.

While the present invention has been described in terms of its specific embodiments, certain modification and equivalents will be apparent to those skilled in the art and are intended.

1. A compound of Formula Ia 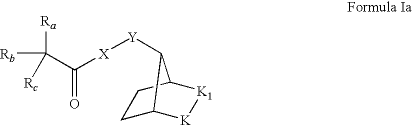
and its pharmaceutically accepted salts, pharmaceutically acceptable solvates, enantiomers, diastereomers, polymorphs or N-oxides
wherein
K is —CH₂ and K₁ is —NR₁; or K₁ is —CH₂ and K is —NR₁;
Y is alkylene or a single bond;
X is O, S or —NRₛ, (wherein Rₛ is selected from hydrogen, alkyl, heteroalkyl, aryl, heteroaryl, heterocyclyl, heteroaryl, heteroarylalkyl, heterocyclylalkyl, or aralkyl);
Rₐ is hydroxy, alkoxy, alkyl or hydrogen;
R_{b} and R_{c} are independently selected from alkyl, alkenyl, alkynyl, aryl, cycloalkyl, heteroaryl, heterocyclyl, aralkyl, heterocyclylalkyl or heteroarylalkyl;
R₁ is hydrogen, alkyl, aryl, heteroaryl, cycloalkyl, heterocyclyl, heteroarylalkyl, heterocyclylalkyl, aralkyl, halogen, carboxy, —C(═O)NRₓR_{y}, —COOR₂, —SO₂R₃, or acyl (wherein R₃, Rₓ, and R_{y} are defined below);
R₂ is selected from alkyl, aryl, aralkyl, heteroaryl, cycloalkyl, heterocyclyl, heterocyclylalkyl or heteroarylalkyl;
R₃ is alkyl, aralkyl, heteroaryl, heterocyclyl, cycloalkyl, heteroaralkyl, heterocyclylalkyl or NRₓR_{y} (wherein Rₓ and R_{y} are defined below);
Rₓ and R_{y} are independently selected from hydrogen, alkyl, cycloalkyl, aryl, halogen, aralkyl, heteroaryl, heterocyclyl, heteroarylalkyl or heterocyclylalkyl; Rₓ and R_{y} may also join together to form a heterocyclyl ring.

2. A compound according to claim 1 selected from the group consisting of
2-Benzyl-2-azabicyclo[2.2.1]hept-7-yl-2-hydroxy(diphenyl)acetate (Compound No. 1),
2-Benzyl-2-azabicyclo[2.2.1]hept-7-yl-2-cyclopentyl(hydroxy)-2-thienylacetate (Compound No. 2),
2-Azabicyclo[2.2.1]hept-7-yl-2-hydroxy(diphenyl)acetate (Compound No. 3),
2-Azabicyclo[2.2.1]hept-7-yl-2-cyclopentyl(hydroxy)phenylacetate (Compound No. 4),
2-Benzyl-2-azabicyclo[2.2.1]hept-7-yl-2-cyclopentyl(hydroxy)phenylacetate (Compound No. 5),
2-Benzyl-2-azabicyclo[2.2.1]hept-7-yl-2-hydroxy(phenyl)-2-thienylacetate (Compound No. 6),
N-(2-benzyl-2-azabicyclo[2.2.1]hept-7-yl)-2,2-diphenylpropanamide (Compound No. 7),
N-(2-benzyl-2-azabicyclo[2.2.1]hept-7-yl)-2-cyclopentyl-2-hydroxy-2-(2-thienyl)acetamide (Compound No. 8),
N-(2-benzyl-2-azabicyclo[2.2.1]hept-7-yl)-2-hydroxy-2-phenyl-2-(2-thienyl)acetamide (Compound No. 9),
N-(2-benzyl-2-azabicyclo[2.2.1]hept-7-yl)-2-cyclopentyl-2-hydroxy-2-phenylacetamide (Compound No. 10),
2-Benzyl-2-azabicyclo[2.2.1]hept-7-yl 2,2-diphenylpropanoate (Compound No. 11),
2-Azabicyclo[2.2.1]hept-7-yl 2,2-diphenylpropanoate (Compound No. 12),
N-(2-benzyl-2-azabicyclo[2.2.1]hept-7-yl)-2-hydroxy-2,2-diphenylacetamide (Compound No. 13),
N-2-azabicyclo[2.2.1]hept-7-yl-2-hydroxy-2,2-diphenylacetamide (Compound No. 14),
N-2-azabicyclo[2.2.1]hept-7-yl-2,2-diphenylpropanamide (Compound No. 15),
N-2-azabicyclo[2.2.1]hept-7-yl-2-cyclopentyl-2-hydroxy-2-phenylacetamide (Compound No. 16),
2-Benzyl-2-azabicyclo[2.2.1]hept-7-yl hydroxy[bis(3-methylphenyl)]acetate (Compound No. 17),
2-Benzyl-2-azabicyclo[2.2.1]hept-7-yl methoxy(diphenyl)acetate (Compound No. 1-8),
(1R)-2-Azabicyclo[2.2.1]hept-7-yl (2R)-((1R or 1S)-3,3-difluorocyclopentyl)(hydroxy)phenylacetate (Compound No. 19),
(1R or 1S)-2-Azabicyclo[2.2.1]hept-7-yl cyclohexyl(hydroxy)phenylacetate (Compound No. 20),
2-(Pyridin-3-ylmethyl)-(1R or 1S)-2-azabicyclo[2.2.1]hept-7-yl cyclopentyl(hydroxy)phenylacetate (Compound No. 21),
2-(1,3-Benzodioxol-5-ylmethyl)-(1R or 1S)-2-azabicyclo[2.2.1]hept-7-yl cyclopentyl(hydroxy)phenylacetate (Compound No. 22),
2-(Cyclohexylmethyl)-(1R or 1S)-2-azabicyclo[2.2.1]hept-7-yl cyclopentyl(hydroxy)phenylacetate (Compound No. 23),
2-(4-Methylbenzyl)-(1R or 1S)-2-azabicyclo[2.2.1]hept-7-yl cyclopentyl(hydroxy)phenylacetate (Compound No. 24),
2-(2-Fluorobenzyl)-(1R or 1S)-2-azabicyclo[2.2.1]hept-7-yl cyclopentyl(hydroxy)phenylacetate (Compound No. 25),
2-(4-Fluorobenzyl)-(1R or 1S)-2-azabicyclo[2.2.1]hept-7-yl cyclopentyl(hydroxy)phenylacetate (Compound No. 26),
2-(3-Methoxybenzyl)-(1R or 1S)-2-azabicyclo[2.2.1]hept-7-yl cyclopentyl(hydroxy)phenylacetate (Compound No. 27),
2-Methyl-(1R or 1S)-2-azabicyclo[2.2.1]hept-7-yl cyclopentyl(hydroxy)phenylacetate (Compound No. 28),
2-(3-Methylbenzyl)-(1R or 1S)-2-azabicyclo[2.2.1]hept-7-yl cyclopentyl(hydroxy)phenylacetate (Compound No. 29),
2-[(1′R)-1-phenylethyl]-(1S or 1R)-2-azabicyclo[2.2.1]hept-7-yl (2R)-cyclopentyl(hydroxy)phenylacetate (Compound No. 30),
(1S or 1R)-2-Azabicyclo[2.2.1]hept-7-yl (2R)-cyclopentyl(hydroxy)phenylacetate (Compound No. 31),
2-[(1′S)-1-phenylethyl]-(1R or 1S)-2-azabicyclo[2.2.1]hept-7-yl cyclohexyl(hydroxy)phenylacetate (Compound No. 32),
2-[(1′S)-1-phenylethyl]-(1R or 1S)-2-azabicyclo[2.2.1]hept-7-yl cyclopentyl(hydroxy)phenylacetate (Compound No. 33),
2-[(1′S)-1-phenylethyl]-(1R or 1S)-2-azabicyclo[2.2.1]hept-7-yl (2R)-[(1S or 1R)
(3,3-difluorocyclopentyl)(hydroxy)phenylacetate (Compound No. 34),
2-[(1′S)-1-phenylethyl]-(1R or 1S)-2-azabicyclo[2.2.1]hept-7-yl (2R)-[(1R or 1S) (3,3-difluorocyclopentyl)](hydroxy)phenylacetate (Compound No. 35),
2-[(1′S)-1-phenylethyl]-(1R or 1S)-2-azabicyclo[2.2.1]hept-7-yl (2R)-cyclopentyl(hydroxy)phenylacetate (Compound No. 36),
(1R or 1S)-2-Azabicyclo[2.2.1]hept-7-yl (2R)-cyclopentyl(hydroxy)phenylacetate (Compound No. 37),
(1R or 1S)-2-Azabicyclo[2.2.1]hept-7-yl cyclopentyl(hydroxy)phenylacetate (Compound No. 38),
(1R or 1S)-7-{[(2R)-2-cyclopentyl-2-hydroxy-2-phenylacetyl]oxy}-2,2-dimethyl-2-azoniabicyclo[2.2.1]heptane Iodide (Compound No. 39)
7-{[cyclopentyl(hydroxy)phenylacetyl]oxy}-(1R or 1S)-2-(4-fluorobenzyl)-2-methyl-2-azoniabicyclo[2.2.1]heptane Iodide (Compound No. 41)
7-{[cyclopentyl(hydroxy)phenylacetyl]oxy}-(1R or 1S)-2-(2-fluorobenzyl)-2-methyl-2-azoniabicyclo[2.2.1]heptane Iodide (Compound No. 42)
7-{[cyclopentyl(hydroxy)phenylacetyl]oxy}-(1R or 1S)-2-methyl-2-(4-methylbenzyl)-2-azoniabicyclo[2.2.1]heptane Iodide (Compound No. 43)
2-(cyclohexylmethyl)-7-{[cyclopentyl(hydroxy)phenylacetyl]oxy}-(1R or 1S)-2-methyl-2-azoniabicyclo[2.2.1]heptane Iodide (Compound No. 44)
7-{[(2R)-2-cyclopentyl-2-hydroxy-2-phenylacetyl]oxy}-(1S or 1R)-2,2-dimethyl-2-azoniabicyclo[2.2.1]heptane Iodide (Compound No. 45)
7-[2-cycloheptyl(hydroxy)-2-thienylacetoxy]-2-methyl-2-[(1′S)-1-phenylethyl]-(1R or 1S)-2-azoniabicyclo[2.2.1]heptane Iodide (Compound No. 46)
7-{[hydroxy(phenyl)-2-thienylacetyl]oxy}-2-methyl-2-[(1′S)-1-phenylethyl]-(1R or 1S)-2-azoniabicyclo[2.2.1]heptane Iodide (Compound No. 47)
7-[2-cycloheptyl(hydroxy)phenylacetoxy]-2-methyl-2-[(1′S)-1-phenylethyl]-(1R or 1S)-2-azoniabicyclo[2.2.1]heptane Iodide (Compound No. 48)
7-[2-hydroxy(diphenyl)acetoxy]-2-methyl-2-[(1′S)-1-phenylethyl]-(1R or 1S)-2-azoniabicyclo[2.2.1]heptane Iodide (Compound No. 49)
7-[2-hydroxy(4-methylphenyl)phenylacetoxy]-2-methyl-2-[(1′S)-1-phenylethyl]-(1R or 1S)-2-azoniabicyclo[2.2.1]heptane Iodide (Compound No. 50)
7-[2-(4-fluorophenyl)(hydroxy)phenylacetoxy]-2-methyl-2-[(1′S)-1-phenylethyl]-(1R or 1S)-2-azoniabicyclo[2.2.1]heptane Iodide (Compound No. 51)
7-{[cyclopentyl(phenyl)acetyl]oxy}-2-methyl-2-[(1′S)-1-phenylethyl]-(1R or 1S)-2-azoniabicyclo[2.2.1]heptane Iodide (Compound No. 52)
7-({(2R)-2-[(1S)-3,3-difluorocyclohexyl]-2-hydroxy-2-phenylacetyl}oxy)-2-methyl-2-[(1′S)-1-phenylethyl]-(1R or 1S)-2-azoniabicyclo[2.2.1]heptane iodide (Compound No. 53)
7-{[(2R)-2-cyclopentyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 54)
7-{[(2R or 2S)-2-cycloheptyl-2-hydroxy-2-phenylacetyl]oxy}-2-methyl-2-[(1′S)-1-phenylethyl]-(1R or 1S)-2-azoniabicyclo[2.2.1]heptane Iodide (Compound No. 55)
2-(1,2,3-benzotrioxol-5-ylmethyl)-7-[(2-cycloheptyl-2-hydroxy-2-phenylacetyl)oxy]-(1R or 1S)-2-methyl-2-azoniabicyclo[2.2.1]heptane Bromide (Compound No. 56)
7-[2-cycloheptyl(hydroxy)phenylacetoxy]-2-ethyl-(1R or 1S)-2-methyl-2-azoniabicyclo[2.2.1]heptane Iodide (Compound No. 57)
7-[2-cycloheptyl(hydroxy)phenylacetoxy]-(1R or 1S)-2-methyl-2-propyl-2-azoniabicyclo[2.2.1]heptane Iodide (Compound No. 58)
7-[2-cycloheptyl(hydroxy)phenylacetoxy]-(1R or 1S)-2-(4-fluorobenzyl)-2-methyl-2-azoniabicyclo[2.2.1]heptane Bromide (Compound No. 59)
7-[2-cycloheptyl(hydroxy)phenylacetoxy]-2-(4-methoxybenzyl)-(1R or 1S)-2-methyl-2-azoniabicyclo[2.2.1]heptane Chloride (Compound No. 60)
7-{[cycloheptyl(hydroxy)phenylacetyl]oxy}-(1R or 1S)-2-methyl-2-(2-methylprop-2-en-1-yl)-2-azoniabicyclo[2.2.1]heptane Bromide (Compound No. 61)
2-benzyl-7-[2-cycloheptyl(hydroxy)phenylacetoxy]-(1R or 1S)-2-methyl-2-azoniabicyclo[2.2.1]heptane Bromide (Compound No. 62)
7-[2-cycloheptyl(hydroxy)phenylacetoxy]-(1R or 1S)-2-methyl-2-(3-phenylpropyl)-2-azoniabicyclo[2.2.1]heptane Bromide (Compound No. 63)
7-({(2R)-2-[(1R)-3,3-difluorocyclopentyl]-2-hydroxy-2-phenylacetyl}oxy)-(1R or 1S)-2-methyl-2-propyl-2-azoniabicyclo[2.2.1]heptane Bromide (Compound No. 64)
7-({(2R)-2-[(1R)-3,3-difluorocyclopentyl]-2-hydroxy-2-phenylacetyl}oxy)-(1R or 1S)-2-methyl-2-(3-phenylpropyl)-2-azoniabicyclo[2.2.1]heptane Bromide (Compound No. 65)
7-({(2R)-2-[(1R)-3,3-difluorocyclopentyl]-2-hydroxy-2-phenylacetyl}oxy)-(1R or 1S)-2-(4-fluorobenzyl)-2-methyl-2-azoniabicyclo[2.2.1]heptane Bromide (Compound No. 66)
7-({(2R)-2-[(1R)-3,3-difluorocyclopentyl]-2-hydroxy-2-phenylacetyl}oxy)-(1R or 1S)-2-methyl-2-(2-methylprop-2-en-1-yl)-2-azoniabicyclo[2.2.1]heptane Bromide (Compound No. 67)
2-(1,3-benzodioxol-5-ylmethyl)-7-({(2R)-2-[(1R)-3,3-difluorocyclopentyl]-2-hydroxy-2-phenylacetyl}oxy)-(1R or 1S)-2-methyl-2-azoniabicyclo[2.2.1]heptane Bromide (Compound No. 68)
7-({(2R)-2-[(1R)-3,3-difluorocyclopentyl]-2-hydroxy-2-phenylacetyl}oxy)-(1R or 1S)-2-(4-methoxybenzyl)-2-methyl-2-azoniabicyclo[2.2.1]heptane Bromide (Compound No. 69)
7-{[(2R)-2-cycloheptyl-2-hydroxy-2-phenylacetyl]oxy-}-(1R or 1S)-2,2-dimethyl-2-azoniabicyclo[2.2.1]heptane Iodide (Compound No. 70)
7-[2-cyclohexyl(hydroxy)phenylacetoxy]-2-methyl-2-[(1S)-1-phenylethyl]-(1R or 1S)-2-azoniabicyclo[2.2.1]heptane Iodide (Compound No. 71)
7-({(2R)-2-[(1S)-3,3-difluorocycloheptyl]-2-hydroxy-2-phenylacetyl}oxy)-(1R or 1S)-2,2-dimethyl-2-azoniabicyclo[2.2.1]heptane Iodide (Compound No. 72)
7-[(2,2-diphenylpropanoyl)oxy]-(1R or 1S)-2,2-dimethyl-2-azoniabicyclo[2.2.1]heptane Iodide (Compound No. 73)
7-({(2R)-2-[(1R)-3,3-difluorocyclohexyl]-2-hydroxy-2-phenylacetyl}oxy)-(1R or 1S)-2,2-dimethyl-2-azoniabicyclo[2.2.1]heptane iodide (Compound No. 74)
7-[2-cycloheptyl(hydroxy)phenylacetoxy]-2-methyl-2-[(1′R)-1-phenylethyl]-(1S or 1R)-2-azoniabicyclo[2.2.1]heptane iodide (Compound No. 75)
7-({(2R)-2-[(1R)-3,3-difluorocyclopentyl]-2-hydroxy-2-phenylacetyl}oxy)-(1R or 1S)-2,2-dimethyl-2-azoniabicyclo[2.2.1]heptane Iodide (Compound No. 76)
7-[2-cycloheptyl(hydroxy)phenylacetoxy]-(1S or 1R)-2,2-dimethyl-2-azoniabicyclo[2.2.1]heptane Iodide (Compound No. 77)
7-{(2R)-2-[(1S)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 78)
7-{(2R)-2-[(1R)-3,3-difluorocyclopentyl](hydroxy)phenylacetoxy}-2-methyl-2-[(1′S)-1-phenylethyl]-(1R or 1S)-2-azoniabicyclo[2.2.1]heptane Iodide (Compound No. 79)
7-{(2R)-[(1R)-3,3-difluorocyclohexyl](hydroxy)phenylacetoxy}-2-methyl-2-[(1′S)-1-phenylethyl]-(1R or 1S)-2-azoniabicyclo[2.2.1]heptane iodide (Compound No. 80)
(1R or 1S)-2-Methyl-2-azabicyclo[2.2.1]hept-7-yl-(2R)-cyclopentyl(hydroxy)phenylacetate (Compound No. 81)
(1S or 1R)-2-Methyl-2-azabicyclo[2.2.1]hept-7-yl-(2R)-cyclopentyl(hydroxy)phenylacetate (Compound No. 82)
(1R or 1S)-2-Methyl-2-azabicyclo[2.2.1]hept-7-yl cyclopentyl(hydroxy)phenylacetate (Compound No. 83)
2-[(1′S)-1-Phenylethyl]-(1R or 1S)-2-azabicyclo[2.2.1]hept-7-yl cycloheptyl(hydroxy)-2-thienylacetate (Compound No. 84)
2-[(1′S)-1-Phenylethyl]-(1R or 1S)-2-azabicyclo[2.2.1]hept-7-yl hydroxy(phenyl)-2-thienylacetate (Compound No. 85)
2-[(1′S)-1-Phenylethyl]-(1R or 1S))-2-azabicyclo[2.2.1]hept-7-yl cycloheptyl(hydroxy)phenylacetate (Compound No. 86)
2-[(1′S)-1-Phenylethyl]-(1R or 1S)-2-azabicyclo[2.2.1]hept-1-yl hydroxy(diphenyl)acetate (Compound No. 87)
2-[(1′S)-1-Phenylethyl]-(1R or 1S)-2-azabicyclo[2.2.1]hept-7-yl hydroxy(4-methylphenyl)phenylacetate (Compound No. 88)
2-[(1′S)-1-Phenylethyl]-(1R or 1S)-2-azabicyclo[2.2.1]hept-7-yl (4-fluorophenyl)(hydroxy)phenylacetate (Compound No. 89)
2-[(1′S)-1-phenylethyl]-(1R or 1S)-2-azabicyclo[2.2.1]hept-7-yl cyclopentyl(phenyl)acetate (Compound No. 90)
2-[(1′S)-1-Phenylethyl]-(1R or 1S)-2-azabicyclo[2.2.1]hept-7-yl-(2R)-[(1S)-3,3-difluorocyclohexyl](hydroxy)phenylacetate (Compound No. 91)
2-[(1′S)-1-phenylethyl]-(1R or 1S)-2-azabicyclo[2.2.1]hept-7-yl (2R)-cyclopentyl(hydroxy)phenylacetate (Compound No. 92)
2-[(1′S)-1-Phenylethyl]-(1R or 1S)-2-azabicyclo[2.2.1]hept-7-yl (2R or 2S)-cycloheptyl(hydroxy)phenylacetate (Compound No. 93)
(1R or 1S)-2-Methyl-2-azabicyclo[2.2.1]hept-7-yl cycloheptyl(hydroxy)phenylacetate (Compound No. 94)
(1R or 1S)-2-Methyl-2-azabicyclo[2.2.1]hept-7-yl (2R)-[(1R)-3,3-difluorocyclopentyl](hydroxy)phenylacetate (Compound No. 95)
2-[(1′S) L-Phenylethyl]-(1R or 1S)-2-azabicyclo[2.2.1]hept-7-yl cyclohexyl(hydroxy)phenylacetate (Compound No. 96)
(1R or 1S)-2-Methyl-2-azabicyclo[2.2.1]hept-7-yl (2R)-[(1S)-3,3-difluorocyclohexyl](hydroxy)phenylacetate (Compound No. 97)
(1R or 1S)-2-Methyl-2-azabicyclo[2.2.1]hept-7-yl 2,2-diphenylpropanoate (Compound No. 98)
(1R or 1S)-2-Methyl-2-azabicyclo[2.2.1]hept-7-yl (2R)-[(1R)-3,3-difluorocyclohexyl](hydroxy)phenylacetate (Compound No. 99)
(1R or 1S)-2-Methyl-2-azabicyclo[2.2.1]hept-7-yl (2R)-[(1R)-3,3-difluorocyclopentyl](hydroxy)phenylacetate (Compound No. 100)
(1R or 1S)-2-Methyl-2-azabicyclo[2.2.1]hept-7-yl cycloheptyl(hydroxy)phenylacetate (Compound No. 101)
2-[(1′S)-1-Phenylethyl]-(1R or 1S)-2-azabicyclo[2.2.1]hept-7-yl-(2R)-[(1S)-3,3-difluorocyclopentyl](hydroxy)phenylacetate (Compound No. 102)
2-[(1′S)-1-phenylethyl]-(1R or 1S)-2-azabicyclo[2.2.1]hept-7-yl-(2R)-[(1R)-3,3-difluorocyclopentyl](hydroxy)phenylacetate (Compound No. 103)
2-[(1′S)-1-phenylethyl]-(1R or 1S)-2-azabicyclo[2.2.1]hept-7-yl-(2R)-[(1R)-3,3-difluorocyclohexyl](hydroxy)phenylacetate (Compound No. 104)
2-[(1′R)-1-Phenylethyl]-(1S or 1R)-2-azabicyclo[2.2.1]hept-7-yl cycloheptyl(hydroxy)phenylacetate (Compound No. 105)

3. A pharmaceutical composition comprising a therapeutically effective amount of a compound as defined in claim 1 together with pharmaceutically acceptable carriers, excipients or diluents.

4. The use of compounds according to claim 1 for the manufacture of medicament for treating or preventing disease or disorder of the respiratory, urinary and gastrointestinal systems, wherein the disease or disorder is mediated through muscarinic receptors in mammals.

5. The use of compounds according to claim 1 for the manufacture of medicament for treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis in mammals.

6. The use of pharmaceutical composition according to claim 3 for the manufacture of medicament for treating or preventing disease or disorder of the respiratory, urinary and gastroinstestinal systems, wherein the disease or disorder is mediated through muscarinic receptors in mammals.

7. The use of pharmaceutical composition according to claim 3 for the manufacture of medicament for treating or preventing urinary incontinence, lower urinary tract symptoms (LUTS), bronchial asthma, chronic obstructive pulmonary disorders (COPD), pulmonary fibrosis, irritable bowel syndrome, obesity, diabetes or gastrointestinal hyperkinesis in mammals.

8. A pharmaceutical composition comprising one or more compounds according to claim 1
and at least one other active ingredients selected from corticosteroids, beta agonists, leukotriene antagonists, 5-lipoxygenase inhibitors, anti-histamines, antitussives, dopamine receptor antagonists, chemokine inhibitors, p38 MAP Kinase inhibitors, and PDE-IV inhibitors.

9. A method of preparing a compound of Formula VI and its pharmaceutically acceptable salts, pharmaceutically acceptable solvates, esters, enantiomers, diastereomers, N-oxides, polymorphs, prodrugs or metabolites,
wherein the reaction comprises of following steps:
a. reacting a compound of Formula I 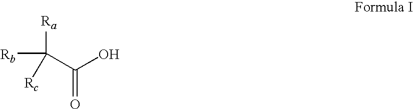
with a compound of Formula II 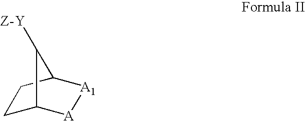
to give a compound of Formula III 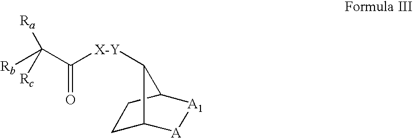
b. deprotecting a compound of Formula III to give a compound of Formula IV 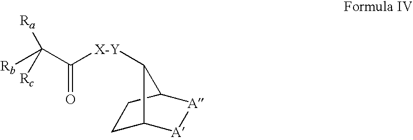
c. N-derivatizing a compound of Formula IV with a compound of Formula Rb-hal to give a compound of Formula IVa 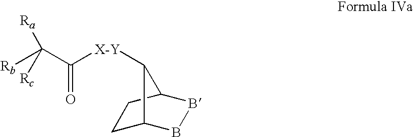
d. reacting a compound of Formula IVa with a compound of Formula Rv-Z₁ to give a compound of Formula VI 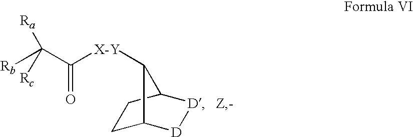
wherein
Rₐ is hydroxy, alkoxy, alkyl or hydrogen;
R_{b} and R_{c} are independently selected from alkyl, alkenyl, alkynyl, aryl, cycloalkyl, heteroaryl, heterocyclyl, aralkyl* heterocyclylalkyl or heteroarylalkyl;
Z₁ is an anion selected from acetate, tartarate, chloride, bromide, iodide, sulphate, phosphate, nitrate, carbonate, fumarate, glutamate, citrate, methanesulphonate, toluenesulphonate, benzenesulphonate, maleate or succinate.
Rv is alkyl, alkenyl, alkynyl, aryl, cycloalkyl, heteroaryl, heterocyclyl, heterocyclylalkyl, heteroarylalkyl or aralkyl.
Y is alkylene or a single bond;
A is —CH₂ and A₁ is —NP; or A₁ is —CH₂ and A is —NP;
A′ is —CH₂ and A″ is —NH; or A″ is CH₂ and A′ is —NH;
B is —CH₂ and B′ is —NRb; or B′ is —CH₂ and B is —NRb;
D is —CH₂ and D′ is 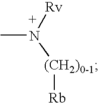
or D′ is —CH₂ and D is

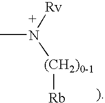

10. A method of preparing a compound of Formula VI and its pharmaceutically acceptable salts, pharmaceutically acceptable solvates, esters, enantiomers, diastereomers, N-oxides, polymorphs, prodrugs or metabolites,
wherein the reaction comprises of following steps:
a. reacting a compound of Formula I 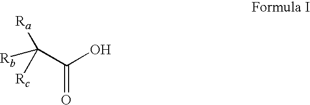
with a compound of Formula II 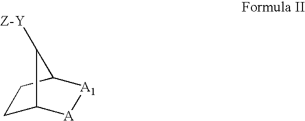
to give a compound of Formula III 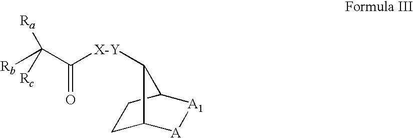
b. deprotecting a compound of Formula III to give a compound of Formula IV 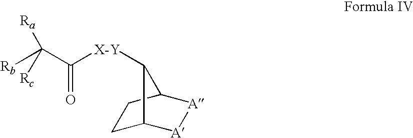
c. N-derivatizing a compound of Formula IV with a compound of Formula Rb—CHO to give a compound of Formula V 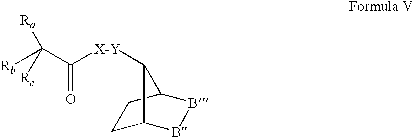
d. reacting a compound of Formula V with a compound of Formula Rv-Z₁ to give a compound of Formula VI 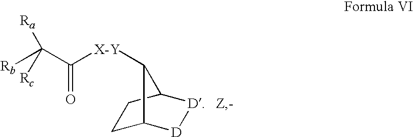
wherein
Rₐ is hydroxy, alkoxy, alkyl or hydrogen;
R_{b} and R_{c} are independently selected from alkyl, alkenyl, alkynyl, aryl, cycloalkyl, heteroaryl, heterocyclyl, aralkyl, heterocyclylalkyl or heteroarylalkyl;
Z₁ is an anion selected from acetate, tartarate, chloride, bromide, iodide, sulphate, phosphate, nitrate, carbonate, fumarate, glutamate, citrate, methanesulphonate, toluenesulphonate, benzenesulphonate, maleate or succinate.
Rv is alkyl, alkenyl, alkynyl, aryl, cycloalkyl, heteroaryl, heterocyclyl, heterocyclylalkyl, heteroarylalkyl or aralkyl.
Y is alkylene or a single bond;
X is O, S or —NRₛ (wherein Rₛ is selected from hydrogen, alkyl, heteroalkyl, aryl, heteroaryl, heterocyclyl, heteroaryl, heteroarylalkyl, heterocyclylalkyl, or aralkyl);
A is —CH₂ and A₁ is —NP; or A₁ is —CH₂ and A is —NP;
A′ is —CH₂ and A″ is —NH; or A″ is CH₂ and A′ is —NH;
B″ is —CH₂ and B′″ is —NCH₂Rb; or B′″ is —CH₂ and B″ is —NCH₂Rb;
D is —CH₂ and D′ is 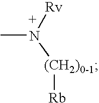
or D′ is —CH₂ and D is [chemical structure]
